(19) United States
(12) Patent Application Publication
(10) Pub. No.: US 2009/0136960 A1
(43) Pub. Date: May 28, 2009

(54) METHODS AND COMPOSITIONS FOR THE IDENTIFICATION OF CANCER MARKERS

(75) Inventors: David M. Lubman, Ann Arbor, MI (US); Diane M. Simeone, Ann Arbor, MI (US); Jia Zhao, Ann Arbor, MI (US)

Correspondence Address: Casimir Jones, S.C., 440 Science Drive, Suite 203, Madison, WI, 53711, US

(73) Assignee: THE REGENTS OF THE UNIVERSITY OF MICHIGAN, Ann Arbor, MI (US)

(21) Appl. No.: 12/294,316

(22) PCT Filed: Mar. 26, 2007
(86) PCT No.: PCT/US07/07409

§ 371 Date: Oct. 22, 2008

Related U.S. Application Data
(60) Provisional application No. 60/785,969, filed on Mar. 24, 2006.

(51) Int. Cl. C12Q 1/68; B01J 19/00; C07K 1/00; B01D 59/44; G01N 33/543; C12Q 1/00

(52) U.S. Cl. 435/6; 422/70; 530/413; 436/518; 435/4; 250/282

(57) ABSTRACT

The present invention relates to methods and compositions for the identification of cancer markers. In particular, the present invention provides methods and compositions for the identification of glycosylated proteins and protein glycosylation patterns. The present invention further provides cancer markers identified using the described methods.

### FIELD OF THE INVENTION

The present invention relates to methods and compositions for the identification of cancer markers. In particular, the present invention provides methods and compositions for the identification of glycosylated proteins and protein glycosylation patterns. The present invention further provides cancer markers identified using the described methods.

### BACKGROUND OF THE INVENTION

Pancreatic cancer is most frequent adenocarcinoma and has the worst prognosis of all cancers, with a five-year survival rate of <3 percent, accounting for the 4^{th} largest number of cancer deaths in the USA (Jemal et al., CA Cancer J Clin., 53: 5-26, 2003). Pancreatic cancer occurs with a frequency of around 9 patients per 100,000 individuals making it the 11^{th} most common cancer in the USA. Currently the only curative treatment for pancreatic cancer is surgery, but only ˜10-20% of patients are candidates for surgery at the time of presentation, and of this group, only ˜20% of patients who undergo a curative operation are alive after five years (Yeo et al., Ann. Surg., 226: 248-257, 1997; Hawes et al., Am. J. Gastroenterol., 95: 17-31, 2000).

The horrible prognosis and lack of effective treatments for pancreatic cancer arise from several causes. Pancreatic cancer tends to rapidly invade surrounding structures and undergo early metastatic spreading, such that it is the cancer least likely to be confined to its organ of origin at the time of diagnosis (Greenlee et al., 2001. CA Cancer J. Clin., 51: 15-36, 2001). Finally, pancreatic cancer is highly resistant to both chemo- and radiation therapies (Greenlee et al., supra). Currently the molecular basis for these characteristics of pancreatic cancer is unknown. What are needed are improved methods for the early diagnosis and treatment of pancreatic cancer. In particular need are serum biomarkers for pancreatic cancer.

### SUMMARY OF THE INVENTION

The present invention relates to methods and compositions for the identification of cancer markers. In particular, the present invention provides methods and compositions for the identification of glycosylated proteins and protein glycosylation patterns. The present invention further provides cancer markers identified using the described methods.

Accordingly, in some embodiments, the present invention provides research methods for the identification of differentially expressed or glycosylated proteins (e.g., in cancer vs. healthy individuals). In other embodiments, the present invention provides methods and compositions for the diagnosis of disease (e.g., cancer) based on the presence of markers identified using the methods of the present invention.

For example, in some embodiments, the present invention provides a system, comprising a lectin affinity chromatography apparatus; and a liquid chromatography apparatus (e.g., a non-porous reverse phase HPLC apparatus) configured to receive a protein sample separated by the lectin affinity chromatography apparatus. In some embodiments, the lectin affinity chromatography apparatus comprises a lectin affinity column including, but not limited to, wheat Germ Agglutinin, Elderberry lectin, and Maackia amurensis lectin. In some embodiments, the system further comprises an apparatus for removal of highly abundant serum proteins (e.g., an IgY-12 proteome partitioning column). In some embodiments, the IgY-12 proteome partitioning column is configured for the removal of albumin, IgG, α1-antitrpsin, IgA, IgM, transferring, haptoglobin, α1-acid glycoprotein, α2-macroglobin, apolipoproteins A-I and A-II and fibrinogen in a single step. In some embodiments, the system further comprises an apparatus for performing polyacrylamide gel electrophoresis. In certain embodiments, the system further comprises a mass spectrometry apparatus (e.g., a MALDI-TOF mass spectrometer, a QIT MALDI quadrupole ion trap-ToF spectrometer, an ESI-TOF mass spectrometer, or an ESI-LTQ mass spectrometer).

In other embodiments, the present invention provides a method, comprising: treating a protein sample with a lectin affinity chromatography apparatus under conditions such that the lectin affinity chromatography apparatus enriches the protein sample for glycosylated proteins to generate a glycosylated protein enriched sample; and separating the glycosylated protein enriched sample with a liquid chromatography apparatus (e.g., a non-porous reverse phase HPLC apparatus) to generate a separated glycosylated enriched protein sample. In some embodiments, the lectin affinity chromatography apparatus comprises a lectin affinity column including, but not limited to, wheat Germ Agglutinin, Elderberry lectin, and Maackia amurensis lectin. In some embodiments, the method further comprises the step of prior to the treating with the lectin affinity chromatography apparatus, the step of treating the protein sample with an apparatus for removal of highly abundant serum proteins (e.g., an IgY-12 proteome partitioning column). In some embodiments, the IgY-12 proteome partitioning column removes albumin, IgG, α1-antitrpsin, IgA, IgM, transferring, haptoglobin, α1-acid glycoprotein, α2-macroglobin, apolipoproteins A-I and A-II and fibrinogen in a single step. In some embodiments, the method further comprises the step of performing polyacrylamide gel electrophoresis (e.g., SDS-PAGE) on the separated glycosylated enriched protein sample. In some preferred embodiments, the method further comprises the step of performing mass spectrometry on the separated glycosylated enriched protein sample (e.g., MALDI-TOF mass spectrometry, QIT MALDI quadrupole ion trap-ToF mass spectrometry, ESI-TOF mass spectrometry, or ESI-LTQ mass spectrometry). In some embodiments, the sample is from a subject diagnosed with cancer.

In yet other embodiments, the present invention provides a method of comparing protein profile maps, comprising treating first and second protein samples with a lectin affinity chromatography apparatus under conditions such that the lectin affinity chromatography apparatus enriches the protein sample for glycosylated proteins to generate first and second glycosylated protein enriched sample; separating the first and second glycosylated protein enriched samples with a liquid chromatography apparatus to generate first and second separated glycosylated enriched protein samples; analyzing the first and second separated glycosylated enriched protein samples with a mass spectrometry apparatus to generate first and second protein profile maps; and comparing the first and second protein profile maps. In some embodiments, the first protein sample is from a subject diagnosed with cancer and wherein the second protein sample is from a cancer free subject. In certain embodiments, the method further comprises the step of identifying proteins that are differentially expressed in the first protein sample relative to the second protein sample. In other embodiments, the method further comprises the step of identifying proteins with altered glycosylation patterns in the first protein sample relative to the second protein sample.

In still further embodiments, the present invention provides a method of diagnosing cancer (e.g., pancreatic cancer) in a subject, comprising: identifying an altered level of expression of a cancer marker selected from the group consisting of plasma protease C1 inhibitor and IgG in a sample from the subject relative to the level in a cancer-free subject. In some embodiments, the cancer marker is expressed at a lower level in a subject with cancer relative to the level in a cancer-free subject. In preferred embodiments, the sample is serum. In some embodiments, the identifying an altered level of expression of the cancer marker comprises identifying an altered level of expression of cancer marker RNA. In other embodiments, the identifying an altered level of expression of the cancer marker comprises identifying an altered level of expression of cancer marker polypeptide.

The present invention additionally provides a method of diagnosing cancer in a subject, comprising: identifying an altered glycosylation pattern of α1-antitrypsin a sample from the subject relative to the glycosylation pattern of the α1-antitrypsin in a cancer-free subject. In some embodiments, identifying an altered glycosylation pattern of α1-antitrypsin comprises analyzing the glycosylation pattern with mass spectrometry, a labeled lectin, a glycosylation specific antibody, or a glycosylation specific reagent.

Additional embodiments of the present invention are described in the description and examples below.

### DESCRIPTION OF THE FIGURES

FIG. 1 shows the strategy used in some embodiments of the present invention to quantify sialylated glycoprotein differences between normal and pancreatic cancer serum and characterize the glycol isoforms and glycan structures.

FIG. 2 shows (a) 2-D gel image of 5 μl non-depleted serum sample stained with Sypro-Ruby dye and (b) pro-Q glycoprotein dye.

FIG. 3 shows (a) UV Chromatogram of 125 μl serum depletion by IgY antibody column to remove 12 high abundant proteins. 20 μg protein from low abundant fraction (b) and 15 ug proteins from high abundant fraction (c) were further separated by a C18 NPS-RP column.

FIG. 4 shows SNA(a), MAL(b) and WGA(c) selected glycoproteins from depleted normal (upper chromatogram) and pancreatic cancer (lower chromatogram). (d) peak a and a′ were further separated by SDS-PAGE gel. Lane 1: peak a from normal serum; Lane 2: peak a′ from cancer serum; Lane 3: MW marker.

FIG. 5 shows MAL(a), SNA(b) and WGA(c) selected glycoproteins from non-depleted normal (upper chromatogram) and pancreatic cancer (lower chromatogram). (d) peak b and b′ were further separated by SDS-PAGE gel. Lane 2: peak b from normal serum; Lane 3: peak b′ from cancer serum; Lane 1: MW marker.

FIG. 6 shows (a) Positive ion MS2 spectrum of the [M+Na]+ ion from biantennary glycan from α1-antitrypsin. (b) MS3 spectrum of Y4 ion (m/z 1298). (c) MS3 spectrum of the B5 ion (m/z 1442). (d) MS4 spectrum [M+Na]+-Y4-m/z 933 (e) MS4 spectrum [M+Na]+-B5-m/z 1077. (f) MS4 spectrum [M+Na]+-B5-m/z 712.

FIG. 7 shows peptide mapping of peak c1 (middle spectrum), c2(top spectrum), c′ (bottom spectrum). (a) Glycopeptide YLGNATAIFFLPDEGK (SEQ ID NO:1) (244-259)+(Hex)5(HexNAc)4(NeuAc)2 (corresponding to #3 in Table 3) (b) Glycopeptide ADTHDEILEGLNFNLTEIPE AQIHEGFQELLR (SEQ ID NO:2) (70-101)+)+(Hex)5(HexNAc)4(NeuAc)2 (#6 in Table 3) (c) Glycopeptide QLAHQSNSTNIFFSPVSIAT AFAMLSLGTK (SEQ ID NO:3) (40-69)+)+(Hex)5(HexNAc)4(NeuAc)2, (#8 in Table 3).

### DEFINITIONS

To facilitate an understanding of the present invention, a number of terms and phrases are defined below:

As used herein, the term “multiphase protein separation” refers to protein separation comprising at least two separation steps. In some embodiments, multiphase protein separation refers to two or more separation steps that separate proteins based on different physical properties of the protein (e.g., a first step that separates based on protein charge and a second step that separates based on protein hydrophobicity).

As used herein, the term “protein profile maps” refers to representations of the protein content of a sample. For example, “protein profile map” includes 2-dimensional displays of total protein expressed in a given cell. In some embodiments, protein profile maps may also display subsets of total protein in a cell. Protein profile maps may be used for comparing “protein expression patterns” (e.g., the amount and identity of proteins expressed in a sample) between two or more samples. Such comparing find use, for example, in identifying proteins that are present in one sample (e.g., a cancer cell) and not in another (e.g., normal tissue), or are over- or under-expressed in one sample compared to the other.

As used herein, the term “2-dimensional protein map” refers to a “protein profile map” that represents (e.g., on two axis of a graph) two properties of the protein content of a sample (e.g., including but not limited to, hydrophobicity and isoelectric point).

As used herein the term “differential display map” and equivalents “differential display plot” and “differential display image” refer to a “protein profile map” that shows the subtraction of one protein profile map from another protein profile map. A differential display map thus shows the differences in proteins present between two samples. A differential display image may also show differences in the abundance of a protein between the two samples. In some embodiments, multiple colors or color gradients are used to represent proteins from each of the two samples.

As used herein, the term “separating apparatus capable of separating proteins based on a physical property” refers to compositions or systems capable of separating proteins (e.g., at least one protein) from one another based on differences in a physical property between proteins present in a sample containing two or more protein species. For example, a variety of protein separation columns and compositions are contemplated including, but not limited to ion exclusion, ion exchange, normal/reversed phase partition, size exclusion, ligand exchange, liquid/gel phase isoelectric focusing, affinity chromatography and adsorption chromatography. These and other apparatuses are capable of separating proteins from one another based on their size, charge, hydrophobicity, and ligand binding affinity, among other properties. A “liquid phase” separating apparatus is a separating apparatus that utilizes protein samples contained in liquid solution, wherein proteins remain solubilized in liquid phase during separation and wherein the product (e.g., fractions) collected from the apparatus are in the liquid phase. This is in contrast to gel electrophoresis apparatuses, wherein the proteins enter into a gel phase during separation. Liquid phase proteins are much more amenable to recovery/extraction of proteins as compared to gel phase. In some embodiments, liquid phase proteins samples may be used in multi-step (e.g., multiple separation and characterization steps) processes without the need to alter the sample prior to treatment in each subsequent step (e.g., without the need for recovery/extraction and resolubilization of proteins).

As used herein, the term “displaying proteins” refers to a variety of techniques used to interpret the presence of proteins within a protein sample. Displaying includes, but is not limited to, visualizing proteins on a computer display representation, diagram, autoradiographic film, list, table, chart, etc. “Displaying proteins under conditions that first and second physical properties are revealed” refers to displaying proteins (e.g., proteins, or a subset of proteins obtained from a separating apparatus) such that at least two different physical properties of each displayed protein are revealed or detectable. For example, such displays include, but are not limited to, tables including columns describing (e.g., quantitating) the first and second physical property of each protein and two-dimensional displays where each protein is represented by an X,Y locations where the X and Y coordinates are defined by the first and second physical properties, respectively, or vice versa. Such displays also include multi-dimensional displays (e.g., three dimensional displays) that include additional physical properties. In some embodiments, displays are generated by “display software.”

As used herein, “characterizing protein samples under conditions such that first and second physical properties are analyzed” refers to the characterization of two or more proteins, wherein two different physical properties are assigned to each analyzed (e.g., displayed, computed, etc.) protein and wherein a result of the characterization is the categorization (i.e., grouping and/or distinguishing) of the proteins based on these two different physical properties. For example, in some embodiments, two proteins are separated based on isoelectric point and hydrophobicity.

As used herein, the term “comparing first and second physical properties of separated protein samples” refers to the comparison of two or more protein samples (or individual proteins) based on two different physical properties of the proteins within each protein sample. Such comparing includes grouping of proteins in the samples based on the two physical properties and comparing certain groups based on just one of the two physical properties (i.e., the grouping incorporates a comparison of the other physical property).

As used herein, the term “delivery apparatus capable of receiving a separated protein from a separating apparatus” refers to any apparatus (e.g., microtube, trough, chamber, etc.) that receives one or more fractions or protein samples from a protein separating apparatus and delivers them to another apparatus (e.g., another protein separation apparatus, a reaction chamber, a mass spectrometry apparatus, etc.).

As used herein, the term “detection system capable of detecting proteins” refers to any detection apparatus, assay, or system that detects proteins derived from a protein separating apparatus (e.g., proteins in one or more fractions collected from a separating apparatus). Such detection systems may detect properties of the protein itself (e.g., UV spectroscopy) or may detect labels (e.g., fluorescent labels) or other detectable signals associated with the protein. The detection system converts the detected criteria (e.g., absorbance, fluorescence, luminescence etc.) of the protein into a signal that can be processed or stored electronically or through similar means (e.g., detected through the use of a photomultiplier tube or similar system).

As used herein, the term “buffer compatible with an apparatus” and “buffer compatible with mass spectrometry” refer to buffers that are suitable for use in such apparatuses (e.g., protein separation apparatuses) and techniques. A buffer is suitable where the reaction that occurs in the presence of the buffer produces a result consistent with the intended purpose of the apparatus or method. For example, a buffer compatible with a protein separation apparatus solubilizes the protein and allows proteins to be separated and collected from the apparatus. A buffer compatible with mass spectrometry is a buffer that solubilizes the protein or protein fragment and allows for the detection of ions following mass spectrometry. A suitable buffer does not substantially interfere with the apparatus or method so as to prevent its intended purpose and result (i.e., some interference may be allowed).

As used herein, the term “automated sample handling device” refers to any device capable of transporting a sample (e.g., a separated or un-separated protein sample) between components (e.g., separating apparatus) of an automated method or system (e.g., an automated protein characterization system). An automated sample handling device may comprise physical means for transporting sample (e.g., multiple lines of tubing connected to a multi-channel valve). In some embodiments, an automated sample handling device is connected to a centralized control network. In some embodiments, the automated sample handling device is a robotic device.

As used herein, the term “switchable multi channel valve” refers to a valve that directs the flow of liquid through an automated sample handling device. The valve preferably has a plurality of channels (e.g., 2 or more, and preferably 4 or more, and more preferably, 6 or more). In addition, in some embodiments, flow to individual channels is “switched” on and off. In some embodiments, valve switching is controlled by a centralized control system. A switchable multi-channel valve allows multiple apparatus to be connected to one automated sample handler. For example, sample can first be directed through one apparatus of a system (e.g., a first chromatography apparatus). The sample can then be directed through a different channel of the valve to a second apparatus (e.g., a second chromatography apparatus).

As used herein, the terms “centralized control system” or “centralized control network” refer to information and equipment management systems (e.g., a computer processor and computer memory) operable linked to multiple devices or apparatus (e.g., automated sample handling devices and separating apparatus). In preferred embodiments, the centralized control network is configured to control the operations or the apparatus an device linked to the network. For example, in some embodiments, the centralized control network controls the operation of multiple chromatography apparatus, the transfer of sample between the apparatus, and the analysis and presentation of data.

As used herein, the terms “computer memory” and “computer memory device” refer to any storage media readable by a computer processor. Examples of computer memory include, but are not limited to, RAM, ROM, computer chips, digital video disc (DVDs), compact discs (CDs), hard disk drives (HDD), and magnetic tape.

As used herein, the term “computer readable medium” refers to any device or system for storing and providing information (e.g., data and instructions) to a computer processor. Examples of computer readable media include, but are not limited to, DVDs, CDs, hard disk drives, magnetic tape and servers for streaming media over networks.

As used herein, the terms “processor” and “central processing unit” or “CPU” are used interchangeably and refers to a device that is able to read a program from a computer memory (e.g., ROM or other computer memory) and perform a set of steps according to the program.

As used herein, the term “hyperlink” refers to a navigational link from one document to another, or from one portion (or component) of a document to another. Typically, a hyperlink is displayed as a highlighted word or phrase that can be selected by clicking on it using a mouse to jump to the associated document or documented portion.

As used herein, the term “display screen” refers to a screen (e.g., a computer monitor) for the visual display of computer generated images. Images are generally displayed by the display screen as a plurality of pixels.

As used herein, the term “computer system” refers to a system comprising a computer processor, computer memory, and a display screen in operable combination. Computer systems may also include computer software.

As used herein, the term “directly feeding” a protein sample from one apparatus to another apparatus refers to the passage of proteins from the first apparatus to the second apparatus without any intervening processing steps. In such a case, the second apparatus “directly receives” the protein sample from the first apparatus. For example, a protein that is directly fed from a protein separating apparatus to a mass spectrometry apparatus does not undergo any intervening digestion steps (i.e., the protein received by the mass spectrometry apparatus is undigested protein).

As used herein, the term “sample” is used in its broadest sense. In one sense it can refer to a cell lysate. In another sense, it is meant to include a specimen or culture obtained from any source, including biological and environmental samples. Biological samples may be obtained from animals (including humans) and encompass fluids, solids, tissues, and gases. Biological samples include blood products (e.g., plasma and serum), saliva, urine, and the like and includes substances from plants and microorganisms. Environmental samples include environmental material such as surface matter, soil, water, and industrial samples. These examples are not to be construed as limiting the sample types applicable to the present invention.

As used herein, the term “subject suspected of having cancer” refers to a subject that presents one or more symptoms indicative of a cancer (e.g., a noticeable lump or mass) or is being screened for a cancer (e.g., during a routine physical). A subject suspected of having cancer may also have one or more risk factors. A subject suspected of having cancer has generally not been tested for cancer. However, a “subject suspected of having cancer” encompasses an individual who has received an initial diagnosis but for whom the stage of cancer is not known. The term further includes people who once had cancer (e.g., an individual in remission).

As used herein, the term “subject at risk for cancer” refers to a subject with one or more risk factors for developing a specific cancer. Risk factors include, but are not limited to, gender, age, genetic predisposition, environmental expose, previous incidents of cancer, preexisting non-cancer diseases, and lifestyle.

As used herein, the term “characterizing cancer in subject” refers to the identification of one or more properties of a cancer sample in a subject, including but not limited to, the presence of benign, pre-cancerous or cancerous tissue, the stage of the cancer, and the subject's prognosis. Cancers may be characterized by the identification of the expression of one or more cancer marker genes, including but not limited to, the cancer markers disclosed herein.

### DETAILED DESCRIPTION OF THE INVENTION

The present invention relates to methods and compositions for the identification of cancer markers. In particular, the present invention provides methods and compositions for the identification of glycosylated proteins and protein glycosylation patterns. The present invention further provides cancer markers identified using the described methods.

Pancreatic cancer is a major oncologic challenge and early detection biomarkers are desperately needed. The biomarker CA19-9 is currently used clinically in patients with pancreatic cancer, however the sensitivity and specificity of the biomarker are not high, and serum levels are significantly increased in inflammatory diseases of the pancreas and biliary tract. More recent RNA-based studies have reported over expression of S100A4, prostate stem cell antigen, osteopontin, mesothelin, hTert and CEACAM1, with elevations of some of these molecules measured in serum, although the clinical applications of these RNA-based markers has not been widely reported (Koopmann et al., Cancer Epidemiol Biomarkers Prev 2004, 13, 487-491; Rosty et al., Am J Pathol 2002, 160, 45-50).

There is currently great interest in developing protein-based serum markers for cancer. Based on the inaccessible location of the pancreas, a serum test is needed to screen patients for the early detection of this disease, particularly in high-risk populations. An important target for serum detection involves the presence of glycosylated proteins. Protein glycosylation has long been recognized as a very common post-translational modification, playing a fundamental role in many biological processes such as immune response and cellular regulation (Bertozzi et al., Science 2001, 291, 2357-2364; Rudd et al., Science 2001, 291, 2370-2376). The glycoproteome is one of the major subproteomes of human serum, where glycoproteins secreted into the blood stream comprise a major part of the serum proteome (Anderson et al., Electrophoresis 1998, 19, 1853-1861). Many clinical biomarkers and therapeutic targets in cancer are glycoproteins, such as CA125 in overian cancer, Her2/neu in breast cancer and prostate-specific antigen in prostate cancer. In addition, the alteration in protein glycosylation which occurs through varying the heterogeneity of glycosylation sites or changing glycan structure of proteins on the cell surface and in body fluids have been shown to correlate with the development of cancer and other disease states (Durand et al., Chem 2000, 46, 795-805). Therefore, a method that can (1) quantitatively analyze glycoprotein abundance and (2) detect the extent of glycosylation alteration and the carbohydrate structure that correlate with pancreatic cancer will be useful for the discovery of new potential diagnostic markers of this disease.

Sialic acids are generally found in the non-reducing terminus of most glycoproteins and glycolipids via a α-2,3 or α-2,6 linkage to galactose or Hex-NAc. Sialic acids are important regulators of cellular and molecular interactions. They can either mask recognition sites or serve as recognition determinants (Kelm et al., Int Rev Cytol 1997, 175, 137-240). Increased sialylation of tumor cell surfaces is well known and is due to either increased activity of the sialyltransferases or due to the increased branching of N-linked carbohydrates leading to termini which can be sialylated (Orntoft et al., Electrophoresis 1999, 20, 362-371). Aberrant sialylation in cancer cells is thought to be a characteristic feature associated with malignant properties including invasiveness and metastatic potential.

Various methods have been developed to enrich glycoproteins. Zhang et al. have developed a method to enrich glycoproteins through hydrazide chemistry (Zhang et al., Nat Biotechnol 2003, 21, 660-666). In this method, the captured glycopeptides were deglycosylated by PNGase F and quantified by isotope labeling. Lectin affinity chromatography has recently been widely used to purify glycoproteins with specific structures. Hancock and coworkers developed a multi-lectin affinity column, which combines ConA, WGA and Jacalin to capture the majority of glycoproteins present in human serum (Yang et al., J Chromatogr A 2004, 1053, 79-88). In related work, Regnier et al utilized serial lectin affinity chromatography (SLAC) for fractionation and comparison of glycan site heterogeneity on glycoproteins derived from human serum (Qiu et al., Anal Chem 2005, 77, 7225-7231; Qiu et al., Anal Chem 2005, 77, 2802-2809). Novotny et al combined silica based lectin microcolumns with high-resolution separation techniques for enrichment of glycoproteins and glycopeptides (Madera et al., Anal Chem 2005, 77, 4081-4090).

In some embodiments, experiments conducted during the course of development of the present invention analyzed pancreatic cancer serum using sialic acid specific lectin affinity chromatography followed by fractionation using RP-HPLC and further separation by SDS-PAGE. The method was used to identify serum marker proteins of pancreatic cancer. The expression of sialic acid glycoproteins with different sub-structures were compared between normal and cancer serum based on UV absorption detection. Low and medium abundant glycoproteins were analyzed after the depletion of 12 highly abundant proteins. Altered glycoproteins were digested and identified by LC-MS/MS. The structures of the released carbohydrate from purified serum proteins were studied using a MALDI-quadrupole-ion trap T of (MALDI-QIT) mass spectrometer. This method was used to detect the change of the isoforms and extent of glycosylation of target glycoproteins in cancer serum. Glyco-peptide mapping was performed using LC-ESI-TOF MS to study the difference of glycosylation efficiency on the glycosylation site of proteins between normal and pancreatic cancer serum. Experiments conducted during the course of development of the present invention identified plasma protease C1 inhibitor and IgG as being down-regulated in serum from patients with pancreatic cancer. Experiments further identified α1-antitrypsin as having an altered glycosylation pattern in serum from pancreatic cancer patients.

### I. Multi-Phase Separation Techniques

In some embodiments, the present invention provides a multi phase separation method (e.g., a lectin chromatography preceded by or followed by additional chromatography steps). The second and subsequence dimensions separate proteins based on a physical property. For example, in some embodiments of the present invention proteins are separated by pI using isoelectric focusing (See e.g., Righetti, Laboratory Techniques in Biochemistry and Molecular Biology; Work, T. S.; Burdon, R. H., Elsevier: Amsterdam, p 10 [1983]). However, the present invention may employ any number of separation techniques including, but not limited to, ion exclusion, ion exchange, normal/reversed phase partition, size exclusion, ligand exchange, liquid/gel phase isoelectric focusing, and adsorption chromatography. In some embodiments (e.g., some automated embodiments), it is preferred that the separations be conducted in the liquid phase to enable products of the separation step to be fed directly into a subsequent liquid phase separation step.

In some embodiments, the proteins collected from the second or subsequent dimensions are identified using proteolytic enzymes, MALDI-TOF MS and MSFit database searching. Certain preferred embodiments are described in detail below. These illustrative examples are not intended to limit the scope of the invention. For example, although the examples are described using human tissues and samples, the methods and apparatuses of the present invention can be used with any desired protein samples including samples from plants and microorganisms.

Exemplary protein separation and analysis methods suitable for use with the present invention are described in more detail below. One skilled in the relevant arts recognizes that additional methods may be utilized. For example, addition protein separation and analysis methods are described, for example, in U.S. Patent applications 20040010126, 20020039747, 20050230315, 20040033591, 20040214233, 20020098595, 20030064527, and U.S. Pat. No. 6,931,325, each of which are herein incorporated by reference in their entirety.

A. Lectin Affinity Chromatography

In some preferred embodiments, lectin affinity is utilized as a first separation step to enrich for glycosylated proteins. Lectins are carbohydrates that bind to glycosylated proteins. The use of lectin affinity chromatography allows for a protein sample to be enriched in glycosylated proteins. The present invention is not limited to the use of lectin affinity chromatography for identifying glycosylation patterns. The present invention contemplates the use of any separation component that separates proteins based on the presence of, type of, or degree of glycosylation, including the use of other affinity columns that recognize sugars or carbohydrate structures.

Lectin affinity columns and chromatography medium are commercially available. For example, in one exemplary embodiment of the present invention, agarose bound lectins wheat Germ Agglutinin, Elderberry lectin, and Maackia amurensis lectin were purchased from Vector Laboratories (Burlingame, Calif., USA). However, the present invention is not limited to the lectin affinity resins described herein. Additional chromatography medium is commercially available. Candidate resins can be evaluated for their ability to bind serum glycoproteins using any suitable method including, but not limited to, those described herein. Protein samples are loaded onto the column and incubated to allow for binding. In some embodiments, non-specifically bound proteins are removed by washing the column with binding buffer. The captured glycoproteins are then released with an elution buffer.

In some embodiments, prior to lectin affinity chromatography, high abundance serum proteins are removed (e.g., using the ProtromeLab IgY-12 proteome partitioning kit (Beckman Coulter, Fullerton, Calif.)). This column enables removal of albumin, IgG, α1-antitrpsin, IgA, IgM, transferring, haptoglobin, α1-acid glycoprotein, α2-macroglobin, HDL (apolipoproteins A-I and A-II) and fibrinogen in a single step. The present invention is not limited to a particular mechanism. Indeed, an understanding of the mechanism is not necessary to practice the present invention. Nonetheless, it is contemplated that the removal of high abundance serum proteins allows for the detection of low abundance proteins that may be masked in the presence of the high abundance proteins.

B. Separation Methods

The following description provides certain preferred embodiments for conducting separation on affinity purified glycosylated proteins according to the methods of the present invention. In some embodiments, affinity purified proteins are separated in one additional separation step. In other embodiments, two or more additional separation steps are utilized.

1. IEF Separation

In some embodiments, the separation is isoelectric focusing. In some embodiments, IEF is performed in a buffer that is compatible with each of the subsequent steps in the separation/analysis methods. Although the present invention provides suitable buffers for use in the particular method configurations described below, one skilled in the art can determine the suitability of a buffer for any particular configuration by solubilizing protein sample in the buffer. If the buffer solubilizes the protein, the sample is run through the particular configuration of separation and detection methods desired. A positive result is achieved if the final step of the desired configuration produces detectable information (e.g., ions are detected in a mass spectrometry analysis). Alternately, the product of each step in the method can be analyzed to determine the presence of the desired product (e.g., determining whether protein elutes from the separation steps).

In some embodiments, n-octyl β-D-glucopyranoside (OG1, from Sigma) is used in the buffer. It is contemplated that detergents of the formula n-octyl SUGARpyranoside find use in these embodiments. The protein solution is loaded to a device that can separate the proteins according to their pI by isoelectric focusing (IEF). In some embodiments, the proteins are solubilized in a running buffer that is compatible with HPLC.

Three exemplary devices that may be used for this step are:

a) Rotofor

This device (Biorad) separates proteins in the liquid phase according to their pI (See e.g., Ayala et al., Appl. Biochem. Biotech. 69:11 [1998]). This device allows for high protein loading and rapid separations that require only four to six hours to perform. Proteins are harvested into liquid fractions after a 5-hour IEF separation. These liquid fractions are ready for analysis by HPLC. This device can be loaded with up to 1 g of protein.

b) Carrier Ampholyte Based Slab Gel IEF Separation with a Whole Gel Eluter

In this case the protein solution is loaded onto a slab gel and the proteins separate in to a series of gel-wide bands containing proteins of the same pI. These proteins are then harvested using a whole gel eluter (WGE, from Biorad). Proteins are then isolated in liquid fractions that are ready for analysis by HPLC. This type of gel can be loaded with up to 20 mg of protein.

c) IPG Slab Gel IEF Separation with a Whole Gel Eluter

Here the proteins are loaded onto a immobiline pI gradient slab gel and separated into a series of gel-wide bands containing proteins of the same pI. These proteins are electro-eluted using the WGE into liquid fractions that are ready for analysis by NP RP HPLC. The IPG gel can be loaded with at least 60 mg of protein.

2. Chromatofocusing

In other embodiments, the separation is chromatofocusing. In chromatofocusing proteins are eluted from the column according to their pH, either one pH unit or fraction thereof, at a time. Columns for chromatofocusing are commercially available (e.g., Mono P HR 5/20 (Amersham Pharmacia, Uppsala, Sweden)). The column is equilibrated with a first buffer to define the upper pH range of the pH gradient. The proteins are then applied. The second focusing buffer is then applied to elute bound proteins, in the order of their isoelectric (pI) points. The pH of the second buffer is lower, and, defines the lower limit of the pH gradient. The pH gradient is formed as the eluting buffer titrates the buffering groups on the ion-exchanger.

3. Protein Separation by NP-RP-HPLC

In some embodiments, subsequence separation steps utilize HPLC (e.g., non-porous reverse phase HPLC). The present invention provides the novel combination of employing non-porous RP packing materials (Eichrom) with another RP HPLC compatible detergent (e.g., n-octyl β-D-galactopyranoside) to facilitate the multi-phase separation of the present invention. This detergent is also compatible with mass spectrometry due to its low molecular weight. The use of these types of RP HPLC columns for protein separations as a second dimension separation after IEF in order to obtain a 2-D protein separation is a novel feature of the present invention. These columns are well suited to this task as the non-porous packing they contain provides optimal protein recovery and rapid efficient separations. It should be noted that though several detergents have been mentioned thus far for increasing protein solubility while being compatible with RP HPLC there are many other different low molecular weight non-ionic detergents that could be used for this purpose. In preferred embodiments, the mobile phase contains a low level of a non-ionic low molecular weight detergent such as n-octyl β-D-glucopyranoside or n-octyl β-D-galactopyranoside as these detergents are compatible with RP HPLC and also with later mass spectrometry analyses (unlike many other detergents); the column should be held at a high temperature (around 60° C.); and the column should be packed with non-porous silica beads to eliminate problems of protein recovery associated with porous packings.

4. PAGE

In some embodiments, polyacrylamide gel electrophoresis (PAGE) is utilized in the separation of protein samples. In some embodiments, SDS-PAGE is utilized. In other embodiments, 2-D gel electrophoresis, where the first dimension separates proteins based on charge, and the second dimension separates proteins based on size, is utilized. Methods for 1-D and 2-D gel electrophoresis are known in the art and include, but are not limited to, those disclosed in the illustrative examples below.

B. Protein Detection and Identification via Mass Spectrometry

In some embodiments of the present invention, following separation, proteins are further characterized using mass spectrometry. For example, in some embodiments, proteins are analyzed by mass spectrometry to determine their molecular weight and identity. The present invention is not limited by the nature of the mass spectrometry technique utilized for such analysis. For example, techniques that find use with the present invention include, but are not limited to, ion trap mass spectrometry, ion trap/time-of-flight mass spectrometry, time of flight/time of flight mass spectrometry, quadrupole and triple quadrupole mass spectrometry, Fourier Transform (ICR) mass spectrometry, and magnetic sector mass spectrometry. The following description of mass spectroscopic analysis and 2-D protein display is illustrated with ESI oa TOF mass spectrometry. Those skilled in the art will appreciate the applicability of other mass spectroscopic techniques to such methods.

For this purpose the proteins eluting from the separation can be analyzed simultaneously to determine molecular weight and identity. A fraction of the effluent is used to determine molecular weight by either MALDI-TOF-MS or ESI oa TOF (LCT, Micromass) (See e.g., U.S. Pat. No. 6,002,127). The remainder of the eluent is used to determine the identity of the proteins via digestion of the proteins and analysis of the peptide mass map fingerprints by either MALDI-TOF-MS or ESI oa TOF. The molecular weight 2-D protein map is matched to the appropriate digest fingerprint by correlating the molecular weight total ion chromatograms (TICs) with the UV-chromatograms and by calculation of the various delay times involved. The UV-chromatograms are automatically labeled with the digest fingerprint fraction number. The resulting molecular weight and digest mass fingerprint data can then be used to search for the protein identity via web-based programs like MSFit (UCSF).

In some embodiments, multiple mass spectrometry (e.g., 2, 3, or more) steps are utilized in the analysis of separated protein fractions. For example, in some embodiments, MALDI-MS/MS is utilized. In other embodiments, MS-MS is utilized.

### II. Differential Display

In some embodiments, the separation methods of the present invention are used to compare expression and/or glycosylation patterns between samples. For example, in some embodiments, expression of glycosylated proteins is compared between samples from a subject diagnosed with cancer and a cancer-free subject. In one illustrative embodiment of the present invention (See e.g., Example 1), the separation methods of the present invention were used to identify markers with differential expression or glycosylation patterns in serum from subjects with pancreatic cancer.

A. Software and Data Presentation

The data generated by the above listed techniques may be presented as 1-D mass maps of intact proteins. In some embodiments, MaxEnt (version 1) software and Mass Lynx version 3.4 (Micromass) are used to analyze mass spectroscopy data. The protein molecular weights are determined by MaxEnt deconvolution of multiply charged protein umbrella mass spectra that are obtained by combining anywhere from 10 to 60 seconds of data from the initial total ion chromatogram (TIC). All deconvoluted mass spectra from a given TIC are added together to produce one mass spectrum for each TIC.

In some embodiments, the data generated in the mass spectroscopy analysis (e.g., TIC's or integrated and deconvoluted mass spectra) are converted to ASCII format and then plotted vertically, using a 256 step gray scale, such that peaks are represented as darkened bands against a white background.

In other embodiments, a color coded 1-D protein profile mass map is generated from differential display of protein molecular weights. In some embodiments, the image is displayed by a computer system as a color-coded mass map, where the intensity of the protein bands corresponds to colors of the rainbow, increasing from blue to green to yellow to red. Thus, the image provides a protein expression pattern that can be used to locate proteins that are differentially displayed in different samples (e.g., cells representing different stages of a cancer). Naturally, the image can be adjusted to show a more detailed zoom of a particular region or the more abundant protein signals can be allowed to saturate thereby showing a clearer image of the less abundant proteins. As the image is automatically digitized it may be readily stored and used to analyze the protein profile of the cells in question. Protein bands on the image can be hyper-linked to other experimental results, obtained via analysis of that band, such as peptide mass fingerprints and MSFit search results. Thus all information obtained about a given 1-D image, including detailed mass spectra, data analyses, and complementary experiments (e.g., immuno-affinity and peptide sequencing) can be accessed from the original image.

The data generated by the above-listed techniques may also be presented as a simple read-out. For example, when two or more samples are compared (e.g., cancerous and non-cancerous cells), the data presented may detail the difference or similarities between the samples (e.g., listing only the proteins that differ in identity or abundance between the samples). In this regard, when the differences between samples (e.g., cancerous and non-cancerous cells) are indicative of a given condition (e.g., cancer cell), the read-out may simply indicate the presence or identity of the condition. In one embodiment, the read-out is a simple +/− indication of the presence of particular proteins or expression patterns associated with a specific condition that is to be analyzed.

A useful feature of the liquid phase method of the present invention is the capability of the high resolution mass spectrometry to quantitate which allows the observer to record relative levels of each form of a given protein. Consequently, it is contemplated that one can determine the relative abundances of a given protein. In addition, post-translational modifications such as differing glycosylation patterns can be found.

With a mass resolution of 5000 Da, a 50000 Da protein can be resolved from a 50010 Da protein. Quantitative comparison between 1-D images can be achieved by spiking samples with known amounts of standard proteins and normalizing images through landmark proteins. Thus, the observer can detect significant abundance changes in the protein profiles of different samples.

B. Presentation of Results

In some preferred embodiments of the present invention, the information generated by the protein profile display is distributed in a coordinated and automated fashion. In some embodiments of the present invention, the data is generated, processed, and/or managed using electronic communications systems (e.g., Internet-based methods).

In some embodiments, a computer-based analysis program is used to translate the raw data generated by the protein profile map (e.g., identity and abundance of proteins in a sample) into data of predictive value for the clinician (e.g., the existence of a malignancy, the probability of pre-cancerous cells becoming malignant, or the type of malignancy). The clinician (e.g., family practitioner or oncologist) can access the predictive data using any suitable means. Thus, in some preferred embodiments, the present invention provides the further benefit that the clinician, who is not likely to be trained in molecular biology or biochemistry, need not understand the raw data of the protein profile map. The data is presented directly to the clinician in its most useful form. The clinician is then able to immediately utilize the information in order to optimize the care of the subject.

The present invention contemplates any method capable of receiving, processing, and transmitting the information to and from medical personal and subject. For example, in some embodiments of the present invention, a sample (e.g., a biopsy) is obtained from a subject and submitted to a protein profiling service (e.g., clinical lab at a medical facility, protein profiling business, etc.) to generate raw data. Once received by the protein profiling service, the sample is processed and a protein profile is produced (i.e., protein expression data), specific for the condition being assayed (e.g., presence of specific cancerous or pre-cancerous cells).

The protein profile data is then prepared in a format suitable for interpretation by a treating clinician. For example, rather than providing raw protein profile data, the prepared format may represent a risk assessment or probability of developing a malignancy that the clinician may use or as recommendations for particular treatment options (e.g., surgery, chemotherapy, or observation). The data may be displayed to the clinician by any suitable method. For example, in some embodiments, the protein profiling service generates a report that can be printed for the clinician (e.g., at the point of care) or displayed to the clinician on a computer monitor.

In some embodiments, the protein profile information (e.g., protein profile map) is first analyzed at a point of care or at a regional facility. The raw data is then sent to a central processing facility for further analysis. The central processing facility provides the advantage of privacy (all data is stored in a central facility with uniform security protocols), speed, and uniformity of data analysis. For example, using an electronic communication system, the central facility can provide data to the clinician, the subject, or researchers. The use of an electronic communications system allows protein profile data to be viewed by clinicians at any location. For example, protein profile data could be accessed by a specialist in the type of disease (e.g., cancer) that the subject is affected with. This allows even remotely located subjects to have their protein profiles analyzed by the leading experts in a particular field. The present invention thus provides a coordinated, timely, and cost effective system for obtaining, analyzing, and distributing life-saving information.

### III. Automation

In some embodiments, all of the above described steps are automated, for example, into one discrete instrument. In one illustrative embodiment, the first dimension is lectin affinity chromatography, with the harvested liquid fractions being directly applied to the second dimension HPLC apparatus through the appropriate tubing. The products from the second dimension separation are then scanned and the data interpreted and displayed as a 2-D representation using the appropriate computer hardware and software. Alternately, the products from the second dimension fractions are sent through the appropriate microtubing to an on-plate MALDI digestion step, followed by mass spectrometry. The resulting data is received and interpreted by a processor. The output data represents any number of desired analyses including, but not limited to, identity of the proteins, mass of the proteins, mass of peptides from protein digests, dimensional displays of the proteins based on any of the detected physical criteria (e.g., size, charge, hydrophobicity, etc.), and the like. In preferred embodiments, the proteins samples are solubilized in a buffer that is compatible with each of the separation and analysis units of the apparatus. Using the automated systems of the present invention provides a protein analysis system that is an order of magnitude less expensive than analogous automation technology for use with 2-D gels (See e.g., Figeys and Aebersold, J. Biomech. Eng. 121:7 [1999]; Yates, J. Mass Spectrom., 33:1 [1998]; and Pinto et al., Electrophoresis 21:181 [2000]).

### IV. Markers for Pancreatic Cancer

As described above, the separation techniques of the present invention were utilized to identify a series of serum pancreatic cancer markers (e.g., plasma protease C1 inhibitor, IgG, and α1-antitrypsin). For example, plasma protease C1 inhibitor and IgG were found to be down-regulated in cancer serum relative to serum from cancer free control subjects. In addition, α1-antitrypsin was found to have an altered glycosylation pattern in cancer serum relative to serum from cancer free controls. In some embodiments, the present invention provides methods of diagnosing pancreatic cancer comprising assaying for the presence of such markers. In preferred embodiments, serum is assayed for altered expression or glycosylation patterns of the markers. In other embodiments, tissue (e.g., biopsy tissue), urine, or blood is assayed.

The present invention is not limited to the markers described above. In some embodiments, additional markers are identified (e.g., using the methods of the present invention).

A. Detection of Markers

In some embodiments, the present invention provides methods for detection of expression of cancer markers (e.g., pancreatic cancer markers). In preferred embodiments, expression is measured directly (e.g., at the RNA or protein level). In some embodiments, expression is detected in tissue samples (e.g., biopsy tissue). In other embodiments, expression is detected in bodily fluids (e.g., including but not limited to, plasma, serum, whole blood, mucus, and urine). The present invention further provides panels and kits for the detection of markers. In preferred embodiments, the presence of a cancer marker is used to provide a prognosis to a subject.

The present invention is not limited to the markers described above. Any suitable marker that correlates with cancer or the progression of cancer may be utilized, including but not limited to, those described in the illustrative examples below (e.g., plasma protease C1 inhibitor, IgG, and α1-antitrypsin). Additional markers are also contemplated to be within the scope of the present invention.

Any suitable method may be utilized to identify and characterize cancer markers suitable for use in the methods of the present invention, including but not limited to, those described in the illustrative Examples below. For example, in some embodiments, markers identified as being up or down-regulated in pancreatic cancer using the methods of the present invention are further characterized using gene expression microarray analysis, tissue microarray, immunohistochemistry, Northern blot analysis, siRNA or antisense RNA inhibition, mutation analysis, investigation of expression with clinical outcome, as well as other methods disclosed herein. Differential glycosylation patterns may be detected by any method, including, but not limited to, mass spectroscopy, antibody affinity, chemical degradation and analysis, and the like.

In some embodiments, the present invention provides a panel for the analysis of a plurality of markers. The panel allows for the simultaneous analysis of multiple markers correlating with carcinogenesis and/or metastasis. For example, a panel may include two or more markers identified as correlating with cancerous tissue, metastatic cancer, localized cancer that is likely to metastasize, pre-cancerous tissue that is likely to become cancerous, chronic pancreatitis, and pre-cancerous tissue that is not likely to become cancerous. Depending on the subject, panels may be analyzed alone or in combination in order to provide the best possible diagnosis and prognosis. Any of the markers described herein may be used in combination with each other or with other known or later identified cancer markers.

In other embodiments, the present invention provides an expression profile map comprising expression profiles of cancers of various stages or prognoses (e.g., likelihood of future metastasis). Such maps can be used for comparison with patient samples. Any suitable method may be utilized, including but not limited to, by computer comparison of digitized data. The comparison data is used to provide diagnoses and/or prognoses to patients.

1. Detection of RNA

In some preferred embodiments, detection of pancreatic cancer markers (e.g., including but not limited to, those disclosed herein) is detected by measuring the expression of corresponding mRNA in a tissue sample (e.g., pancreatic tissue). mRNA expression may be measured by any suitable method, including but not limited to, those disclosed below.

In some embodiments, RNA is detected by Northern blot analysis. Northern blot analysis involves the separation of RNA and hybridization of a complementary labeled probe.

In still further embodiments, RNA (or corresponding cDNA) is detected by hybridization to an oligonucleotide probe). A variety of hybridization assays using a variety of technologies for hybridization and detection are available. For example, in some embodiments, the TaqMan assay (PE Biosystems, Foster City, Calif.; See e.g., U.S. Pat. Nos. 5,962,233 and 5,538,848, each of which is herein incorporated by reference) is utilized. The assay is performed during a PCR reaction. The TaqMan assay exploits the 5′-3′ exonuclease activity of the AMPLITAQ GOLD DNA polymerase. A probe consisting of an oligonucleotide with a 5′-reporter dye (e.g., a fluorescent dye) and a 3′-quencher dye is included in the PCR reaction. During PCR, if the probe is bound to its target, the 5′-3′ nucleolytic activity of the AMPLITAQ GOLD polymerase cleaves the probe between the reporter and the quencher dye. The separation of the reporter dye from the quencher dye results in an increase of fluorescence. The signal accumulates with each cycle of PCR and can be monitored with a fluorimeter.

In yet other embodiments, reverse-transcriptase PCR (RT-PCR) is used to detect the expression of RNA. In RT-PCR, RNA is enzymatically converted to complementary DNA or “cDNA” using a reverse transcriptase enzyme. The cDNA is then used as a template for a PCR reaction. PCR products can be detected by any suitable method, including but not limited to, gel electrophoresis and staining with a DNA specific stain or hybridization to a labeled probe. In some embodiments, the quantitative reverse transcriptase PCR with standardized mixtures of competitive templates method described in U.S. Pat. Nos. 5,639,606, 5,643,765, and 5,876,978 (each of which is herein incorporated by reference) is utilized.

2. Detection of Protein

In other embodiments, gene expression of cancer markers is detected by measuring the expression of the corresponding protein or polypeptide. Protein expression may be detected by any suitable method. In some embodiments, proteins are detected by immunohistochemistry. In other embodiments, proteins are detected by their binding to an antibody raised against the protein. The generation of antibodies is described below.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the presence or absence of a series of proteins corresponding to cancer markers is utilized.

In other embodiments, the immunoassay described in U.S. Pat. Nos. 5,599,677 and 5,672,480; each of which is herein incorporated by reference is utilized.

3. Detection of Glycosylation Patterns

In some embodiments, the presence of glycosylated proteins or protein glycosylation patterns is detected using standard protein detection methods (e.g., those described above). In other embodiments, differences in glycosylation patterns are detected using glycosylation specific methods. For example, in some embodiments, the mass spectrometry methods described herein are utilized to analyze the glycosylation pattern of a specific cancer marker protein. In other embodiments, glycosylation specific reagents (e.g., including, but not limited to, biotinylated or otherwise labeled lectins, glycosylation specific antibodies, or periodic acid-schiff detection methods) are utilized. Reagents for such assays are commercially available.

In some embodiments, a computer-based analysis program is used to translate the raw data generated by the detection assay (e.g., the presence, absence, or amount of a given marker or markers) into data of predictive value for a clinician (See e.g., the above description of data analysis and distribution methods).

4. Kits

In yet other embodiments, the present invention provides kits for the detection and characterization of pancreatic cancer. In some embodiments, the kits contain antibodies specific for a cancer marker, in addition to detection reagents and buffers. In other embodiments, the kits contain reagents specific for the detection of mRNA or cDNA (e.g., oligonucleotide probes or primers). In still further embodiments, the kits contain reagents for identifying glycosylated protein (e.g., the glycosylation detection reagents described above). In preferred embodiments, the kits contain all of the components necessary to perform a detection assay, including all controls, directions for performing assays, and any necessary or desired software for analysis and presentation of results.

6. In Vivo Imaging

In some embodiments, in vivo imaging techniques are used to visualize the expression of cancer markers in an animal (e.g., a human or non-human mammal). For example, in some embodiments, cancer marker mRNA or protein is labeled using a labeled antibody specific for the cancer marker. A specifically bound and labeled antibody can be detected in an individual using an in vivo imaging method, including, but not limited to, radionuclide imaging, positron emission tomography, computerized axial tomography, X-ray or magnetic resonance imaging method, fluorescence detection, and chemiluminescent detection. Methods for generating antibodies to the cancer markers of the present invention are described below.

The in vivo imaging methods of the present invention are useful in the diagnosis of cancers that express the cancer markers of the present invention (e.g., pancreatic cancer). In vivo imaging is used to visualize the presence of a marker indicative of the cancer. Such techniques allow for diagnosis without the use of an unpleasant biopsy. The in vivo imaging methods of the present invention are also useful for providing prognoses to cancer patients. For example, the presence of a marker indicative of cancers likely to metastasize can be detected. The in vivo imaging methods of the present invention can further be used to detect metastatic cancers in other parts of the body.

In some embodiments, reagents (e.g., antibodies) specific for the cancer markers of the present invention are fluorescently labeled. The labeled antibodies are introduced into a subject (e.g., orally or parenterally). Fluorescently labeled antibodies are detected using any suitable method (e.g., using the apparatus described in U.S. Pat. No. 6,198,107, herein incorporated by reference).

In other embodiments, antibodies are radioactively labeled. The use of antibodies for in vivo diagnosis is well known in the art. Sumerdon et al., (Nucl. Med. Biol 17:247-254 [1990] have described an optimized antibody-chelator for the radioimmunoscintographic imaging of tumors using Indium-111 as the label. Griffin et al., (J Clin One 9:631-640 [1991]) have described the use of this agent in detecting tumors in patients suspected of having recurrent colorectal cancer. The use of similar agents with paramagnetic ions as labels for magnetic resonance imaging is known in the art (Lauffer, Magnetic Resonance in Medicine 22:339-342 [1991]). The label used will depend on the imaging modality chosen. Radioactive labels such as Indium-111, Technetium-99m, or Iodine-131 can be used for planar scans or single photon emission computed tomography (SPECT). Positron emitting labels such as Fluorine-19 can also be used for positron emission tomography (PET). For MRI, paramagnetic ions such as Gadolinium (III) or Manganese (II) can be used.

Radioactive metals with half-lives ranging from 1 hour to 3.5 days are available for conjugation to antibodies, such as scandium-47 (3.5 days) gallium-67 (2.8 days), gallium-68 (68 minutes), technetiium-99m (6 hours), and indium-111 (3.2 days), of which gallium-67, technetium-99m, and indium-111 are preferable for gamma camera imaging, gallium-68 is preferable for positron emission tomography.

A useful method of labeling antibodies with such radiometals is by means of a bifunctional chelating agent, such as diethylenetriaminepentaacetic acid (DTPA), as described, for example, by Khaw et al. (Science 209:295 [1980]) for In-111 and Tc-99m, and by Scheinberg et al. (Science 215:1511 [1982]). Other chelating agents may also be used, but the 1-(p-carboxymethoxybenzyl) EDTA and the carboxycarbonic anhydride of DTPA are advantageous because their use permits conjugation without affecting the antibody's immunoreactivity substantially.

Another method for coupling DPTA to proteins is by use of the cyclic anhydride of DTPA, as described by Hnatowich et al. (Int. J. Appl. Radiat. Isot. 33:327 [1982]) for labeling of albumin with In-111, but which can be adapted for labeling of antibodies. A suitable method of labeling antibodies with Tc-99m, which does not use chelation with DPTA, is the pretinning method of Crockford et al., (U.S. Pat. No. 4,323,546, herein incorporated by reference).

A preferred method of labeling immunoglobulins with Tc-99m is that described by Wong et al. (Int. J. Appl. Radiat. Isot., 29:251 [1978]) for plasma protein, and recently applied successfully by Wong et al. (J. Nucl. Med., 23:229 [1981]) for labeling antibodies.

In the case of the radiometals conjugated to the specific antibody, it is likewise desirable to introduce as high a proportion of the radiolabel as possible into the antibody molecule without destroying its immunospecificity. A further improvement may be achieved by effecting radiolabeling in the presence of the specific cancer marker of the present invention, to insure that the antigen-binding site on the antibody will be protected. The antigen is separated after labeling.

In still further embodiments, in vivo biophotonic imaging (Xenogen, Almeda, Calif.) is utilized for in vivo imaging. This real-time in vivo imaging utilizes luciferase. The luciferase gene is incorporated into cells, microorganisms, and animals (e.g., as a fusion protein with a cancer marker of the present invention). When active, it leads to a reaction that emits light. A CCD camera and software is used to capture the image and analyze it.

B. Antibodies

The present invention provides isolated antibodies. In preferred embodiments, the present invention provides monoclonal antibodies that specifically bind to an isolated polypeptide comprised of at least five amino acid residues of the cancer markers described herein. These antibodies find use in the diagnostic methods described herein.

An antibody against a protein of the present invention may be any monoclonal or polyclonal antibody, as long as it can recognize the protein. Antibodies can be produced by using a protein of the present invention as the antigen according to a conventional antibody or antiserum preparation process.

The present invention contemplates the use of both monoclonal and polyclonal antibodies. Any suitable method may be used to generate the antibodies used in the methods and compositions of the present invention, including but not limited to, those disclosed herein. For example, for preparation of a monoclonal antibody, protein, as such, or together with a suitable carrier or diluent is administered to an animal (e.g., a mammal) under conditions that permit the production of antibodies. For enhancing the antibody production capability, complete or incomplete Freund's adjuvant may be administered. Normally, the protein is administered once every 2 weeks to 6 weeks, in total, about 2 times to about 10 times. Animals suitable for use in such methods include, but are not limited to, primates, rabbits, dogs, guinea pigs, mice, rats, sheep, goats, etc.

For preparing monoclonal antibody-producing cells, an individual animal whose antibody titer has been confirmed (e.g., a mouse) is selected, and 2 days to 5 days after the final immunization, its spleen or lymph node is harvested and antibody-producing cells contained therein are fused with myeloma cells to prepare the desired monoclonal antibody producer hybridoma. Measurement of the antibody titer in antiserum can be carried out, for example, by reacting the labeled protein, as described hereinafter and antiserum and then measuring the activity of the labeling agent bound to the antibody. The cell fusion can be carried out according to known methods, for example, the method described by Koehler and Milstein (Nature 256:495 [1975]). As a fusion promoter, for example, polyethylene glycol (PEG) or Sendai virus (HVJ), preferably PEG is used.

Examples of myeloma cells include NS-1, P3U1, SP2/0, AP-1 and the like. The proportion of the number of antibody producer cells (spleen cells) and the number of myeloma cells to be used is preferably about 1:1 to about 20:1. PEG (preferably PEG 1000-PEG 6000) is preferably added in concentration of about 10% to about 80%. Cell fusion can be carried out efficiently by incubating a mixture of both cells at about 20° C. to about 40° C., preferably about 30° C. to about 37° C. for about 1 minute to 10 minutes.

Various methods may be used for screening for a hybridoma producing the antibody (e.g., against a cancer marker of the present invention). For example, where a supernatant of the hybridoma is added to a solid phase (e.g., microplate) to which antibody is adsorbed directly or together with a carrier and then an anti-immunoglobulin antibody (if mouse cells are used in cell fusion, anti-mouse immunoglobulin antibody is used) or Protein A labeled with a radioactive substance or an enzyme is added to detect the monoclonal antibody against the protein bound to the solid phase. Alternately, a supernatant of the hybridoma is added to a solid phase to which an anti-immunoglobulin antibody or Protein A is adsorbed and then the protein labeled with a radioactive substance or an enzyme is added to detect the monoclonal antibody against the protein bound to the solid phase.

Selection of the monoclonal antibody can be carried out according to any known method or its modification. Normally, a medium for animal cells to which HAT (hypoxanthine, aminopterin, thymidine) are added is employed. Any selection and growth medium can be employed as long as the hybridoma can grow. For example, RPMI 1640 medium containing 1% to 20%, preferably 10% to 20% fetal bovine serum, GIT medium containing 1% to 10% fetal bovine serum, a serum free medium for cultivation of a hybridoma (SFM-101, Nissui Seiyaku) and the like can be used. Normally, the cultivation is carried out at 20° C. to 40° C., preferably 37° C. for about 5 days to 3 weeks, preferably 1 week to 2 weeks under about 5% CO₂ gas. The antibody titer of the supernatant of a hybridoma culture can be measured according to the same manner as described above with respect to the antibody titer of the anti-protein in the antiserum.

Separation and purification of a monoclonal antibody (e.g., against a cancer marker of the present invention) can be carried out according to the same manner as those of conventional polyclonal antibodies such as separation and purification of immunoglobulins, for example, salting-out, alcoholic precipitation, isoelectric point precipitation, electrophoresis, adsorption and desorption with ion exchangers (e.g., DEAE), ultracentrifugation, gel filtration, or a specific purification method wherein only an antibody is collected with an active adsorbent such as an antigen-binding solid phase, Protein A or Protein G and dissociating the binding to obtain the antibody.

Polyclonal antibodies may be prepared by any known method or modifications of these methods including obtaining antibodies from patients. For example, a complex of an immunogen (an antigen against the protein) and a carrier protein is prepared and an animal is immunized by the complex according to the same manner as that described with respect to the above monoclonal antibody preparation. A material containing the antibody against is recovered from the immunized animal and the antibody is separated and purified.

As to the complex of the immunogen and the carrier protein to be used for immunization of an animal, any carrier protein and any mixing proportion of the carrier and a hapten can be employed as long as an antibody against the hapten, which is crosslinked on the carrier and used for immunization, is produced efficiently. For example, bovine serum albumin, bovine cycloglobulin, keyhole limpet hemocyanin, etc. may be coupled to an hapten in a weight ratio of about 0.1 part to about 20 parts, preferably, about 1 part to about 5 parts per 1 part of the hapten.

In addition, various condensing agents can be used for coupling of a hapten and a carrier. For example, glutaraldehyde, carbodiimide, maleimide activated ester, activated ester reagents containing thiol group or dithiopyridyl group, and the like find use with the present invention. The condensation product as such or together with a suitable carrier or diluent is administered to a site of an animal that permits the antibody production. For enhancing the antibody production capability, complete or incomplete Freund's adjuvant may be administered. Normally, the protein is administered once every 2 weeks to 6 weeks, in total, about 3 times to about 10 times.

The polyclonal antibody is recovered from blood, ascites and the like, of an animal immunized by the above method. The antibody titer in the antiserum can be measured according to the same manner as that described above with respect to the supernatant of the hybridoma culture. Separation and purification of the antibody can be carried out according to the same separation and purification method of immunoglobulin as that described with respect to the above monoclonal antibody.

The protein used herein as the immunogen is not limited to any particular type of immunogen. For example, a cancer marker of the present invention (further including a gene having a nucleotide sequence partly altered) can be used as the immunogen. Further, fragments of the protein may be used. Fragments may be obtained by any methods including, but not limited to expressing a fragment of the gene, enzymatic processing of the protein, chemical synthesis, and the like.

### EXPERIMENTAL

The following examples serve to illustrate certain preferred embodiments and aspects of the present invention and are not to be construed as limiting the scope thereof.

### Example 1

### Materials and Methods

### Samples:

Human normal serum and pancreatic cancer serum were provided by University Hospital. 40 cc of blood was provided by each patient. The samples were permitted to sit at room temperature for a minimum of 30 minutes (and a maximum of 60 minutes) to allow the clot to form in the red top tubes, then centrifuged at 1,300×g at 4° C. for 20 minutes. The serum was removed, transferred to a polypropylene capped tube and frozen. The frozen samples were stored at −70° C. until assayed. Six samples (three normal serum and three cancer serum) were studied in this work.

### Removing High Abundant Proteins Using Antibody Column and Protein Assay

125 μl of human serum was depleted using the ProtromeLab IgY-12 proteome partitioning kit (Beckman Coulter, Fullerton, Calif.) after brief centrifugation using a 0.45 μm spin filter for 1 min at 9200×g. The experimental procedure follows the protocol provided by Beckman. This column enables removal of albumin, IgG, α1-antitrpsin, IgA, IgM, transferring, haptoglobin, α1-acid glycoprotein, α2-macroglobin, HDL (apolipoproteins A-I and A-II) and fibrinogen in a single step. The final volume of serum sample in elution buffer after depletion is 15-20 ml. This volume was concentrated using 15 ml, 10 kDa Amicon filters (Millipore, Billerica, Mass.).

Protein assays were carried out in a 250 μl transparent 96 well plate (Fisher, Barrington, Ill.) according to the Bradford assay method since the plate based method requires less sample than the cuvette based assay and it enables the simultaneous reading of all the samples and standards.

### MAL, SNA and WGA Affinity Selection

Agarose bound lectins, Wheat Germ Agglutinin, (WGA) Elderberry lectin, (SNA), and Maackia amurensis lectin, (MAL) were purchased from Vector Laboratories (Burlingame, Calif., USA). Agarose bound WGA was packed into the disposal screw end-cap spin column with filters at both ends. The column was first washed with 500 μl binding buffer (20 mM Tris, 0.2M NaCl, pH7.4) by centrifuging the spin column at 500 rpm for 2 min. The protease inhibitor stock solution was prepared by dissolving one complete EDTA-free Protease inhibitor cocktail tablet (Roche, Indianapolis, Ind.) in 1 ml H₂O. The stock solution was added to binding buffer and elution buffer at a ratio of 1:50. 50 μl depleted or non-depleted serum sample diluted with 500 μl binding buffer was loaded onto the column and incubated for 15 min. The column was centrifuged for 2 min at 500 rpm to remove the non-binding fraction. The column was washed with 600 μl binding buffer twice to wash off the non-specific binding. The captured glycoproteins were released with 150 μl elution buffer (0.5M N-acetyl-glucosamine in 20 mM Tris and 0.5 M NaCl, pH 7.0) and the eluted fraction was collected by centrifugation at 500 rpm for 2 min. This step was repeated twice and the eluate fractions were pooled.

SNA and MAL spin columns were purchased from QIAGEN (Valencia, Calif.) and the elution procedure was similar to that used with the WGA spin column. The elution buffer for these two lectins is 0.3 M lactose in buffered saline.

### RP-HPLC Separation of Lectin Bound Glycoproteins

The enriched glycoprotein fraction was loaded onto nonporous silica reverse phase high-performance liquid chromatography (NPS-RP-HPLC) for separation. High separation efficiency was achieved by using an ODSIII-E (4.6×33 mm) column (Eprogen, Inc., Darien, Ill.) packed with 1.5 μm non-porous silica. To collect purified proteins from NPS-RP-HPLC, the reversed-phase separation was performed at 0.5 mL/min and monitored at 214 nm using a Beckman 166 Model UV detector (Beckman-Coulter). Proteins eluting from the column were collected by an automated fraction collector (Model SC 100; Beckman-coulter), controlled by an in-house designed DOS-based software program. To enhance the speed, resolution and reproducibility of the separation, the reversed phase column was heated to 60° C. by a column heater (Jones Chromatography, Model 7971). Both mobile phase A (water) and B (ACN) contained 0.1% v/v TFA. The gradient profile used was as follows: 5% to 15% B in 1 min, 15% to 25% B in 2 min, 25% to 30% B in 3 min, 30% to 41% B in 15 min, 41% to 47% B in 4 min, 47% to 67% B in 5 min and 67% to 100% B in 2 min. Deionized water was purified using a Millipore RG system (Bedford, Mass.).

### Gel Electrophoresis and Fluorescence Dye Labeling

### SDS-PAGE:

The fractions collected from RP-HPLC were further separated by SDS-PAGE according to Laemmli (Laemmli, Nature 1970, 227, 680-685), run in a Mini-PROTEAN Cell (Bio-Rad, Hercules, Calif.) at 80 volts controlled by Power Pac3000 (Bio-Rad, Hercules, Calif.). The proteins were visualized by staining with Sypro-ruby fluorescence dye (Molecular Probes, Carlsbad, Calif.). The staining was performed according to the protocol suggested by the manufacturer.

### 2-D PAGE:

2-D electrophoresis was performed according to “2-D gel electrophoresis principles and methods” (Amersham, Piscataway, N.J.). 5 μl serum sample was loaded in a 11 cm (pH 3-10) IPG gel (Bio-rad). The first dimension separation was carried out on a Protean IEF Cell (Bio-rad) with a maximum of 35000 vhr. 4-20% poly-acrylamide gel (11×16 cm) was used for the second dimension separation, which was carried out in a Hoefer SE600 electrophoresis unit (Amersham). The 2-D gel was first stained with pro-Q glycoprotein dye (Molecular Probes, Carlsbad, Calif.) followed by Sypro-Ruby fluorescence dye staining. The staining procedure of these two dyes follows the protocol provided.

### Protein Digestion by Trypsin

Fractions obtained from NPS-RP-HPLC were concentrated down to ˜20 uL using a SpeedVac concentrator (Thermo, Milford, Mass.) operating at 45° C. 20 μl of 100 mM ammonium bicarbonate (Sigma) was then mixed with each concentrated sample to obtain a pH value of ˜7.8. 0.5 μl of TPCK modified sequencing grade porcine trypsin (Promega, Madison, Wis.) was added and vortexed prior to a 12-16 hour incubation at 37° C. on an agitator. For in-gel digestion, a gel slice was destained in 200 mM NH₄HCO₃ in 40% ACN and incubated at 37° C. for 30 mins. After reduction and alkylation, gel pieces were dried down in a speedvac. 50 μl reaction solution (100 mM NH₄HCO₃ in 9% ACN) and 1 μl trypsin (Promega) were added to the gel sample. After 12-16 h incubation at 37° C., the liquid from the gel piece was removed and transferred to a new tube.

### Glycan Cleavage by PNGase F and Glycan Purification

For glycan cleavage and purification, the procedure follows that of Yu et al. (Rapid Commun Mass Spectrom 2005, 19, 2331-2336). The peaks collected from NPS RP-HPLC were dried down completely and redissolved in 40 μl 0.1% (w/v) RapiGest solution (Waters, Milford, Mass.) prepared in 50 mM NH₄HCO₃ buffer, pH 7.9 to denature the protein. Protein samples were reduced with 5 mM DTT for 45 min at 56° C. and alkylated with 15 mM iodoacetamide in the dark for 1 h at room temperature. 2 μl enzyme PNGase F (New England Biolabs, Ipswich, Mass.) was added to the samples and the solution was incubated for 24 h at 37° C. The glycans released were purified prior to MALDI-MS analysis using SPE micro-elution plates (Waters) packed with HILIC sorbent (5 mg). Salt, protein and detergent were removed at this step. The micro-elution SPE device was operated using a centrifugation device with a plate adaptor (Thermo).

### Mass Spectrometry

### Glycan Structure Analysis

MS and MSn spectra of glycan samples were acquired on a Shimadzu Axima QIT MALDI quadrupole ion trap-ToF (MALDI-QIT) (Manchester, UK). Acquisition and data processing were controlled by Launch-pad software (Karatos, Manchester, UK). A pulsed N2 laser light (337 nm) with a pulse rate of 5 Hz was used for ionization. Each profile results from 2 laser shots. Argon was used as the collision gas for CID and helium was used for cooling the trapped ions. The TOF was externally calibrated using 500 fmol/μl of bradykinin fragment 1-7 (757.40 m/z), angiotensin II (1046.54 m/z), P14R (1533.86 m/z), ACTH (2465.20 m/z) (sigma). 25 mg/ml 2,5-dihydroxybenzonic acid (DHB) (LaserBio Labs, France) was prepared in 50% ACN with 0.1% TFA. 0.5 μl glycan sample was spotted on the stainless-steel target and 0.5 ul matrix solution was added followed by air drying.

### Glycopeptide Mapping

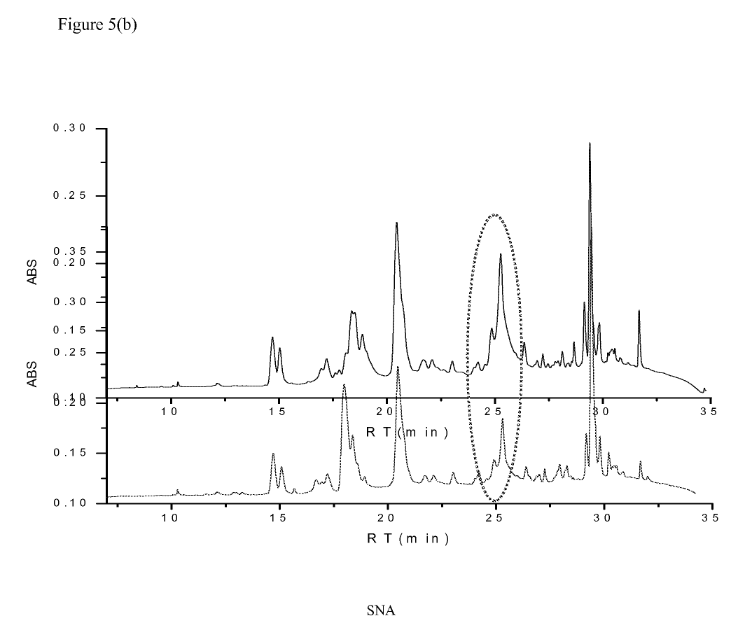

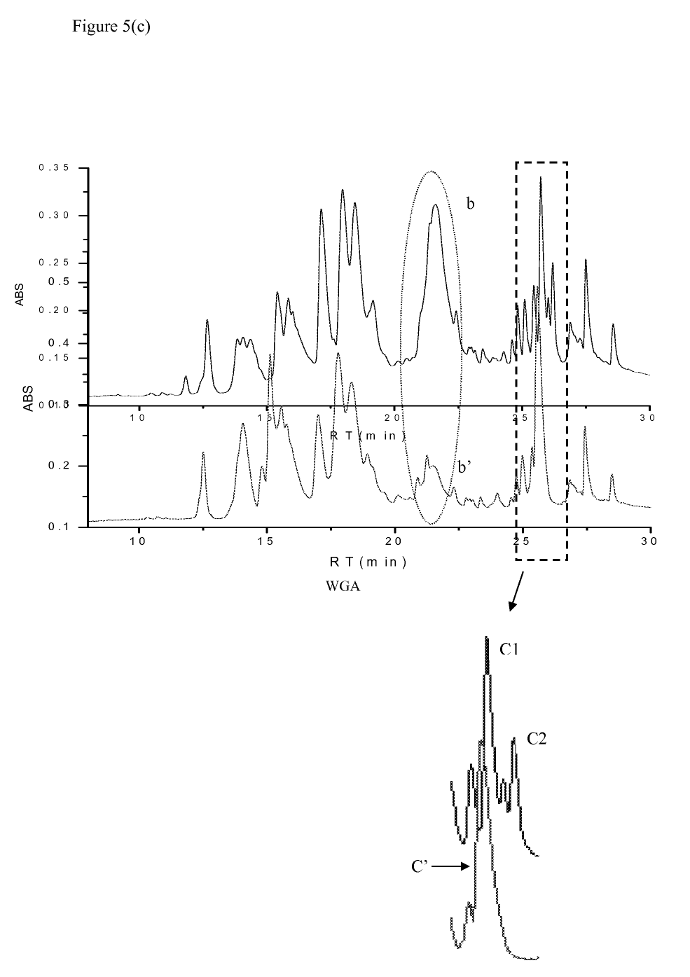

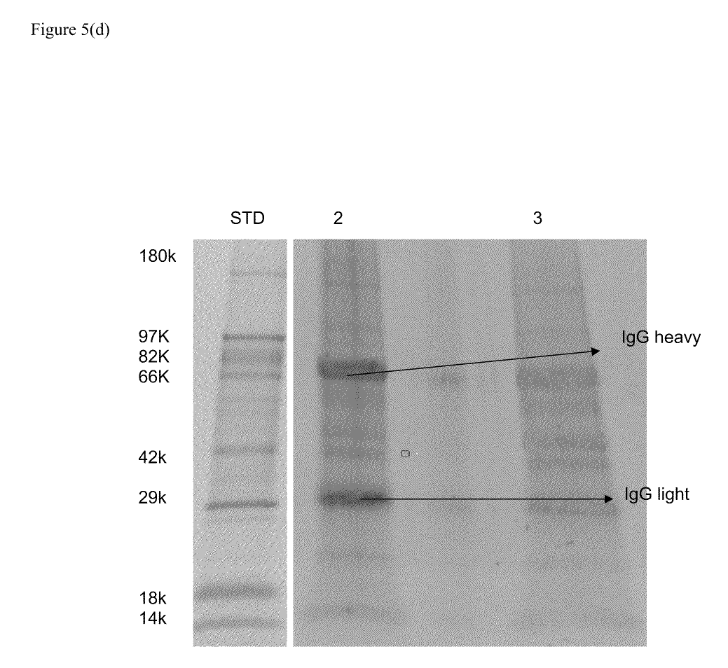

Digested peptide mixtures from peak c1, c2 and c′ in FIG. 5 were separated by a capillary RP column (C18, 0.3×50 mm) (Michrom, Auburn, Calif.) on a Paradigm MG4 micro-pump (Michrom) with a flow rate of 5 μl/min. The gradient starts at 5% ACN, was ramped to 60% ACN in 25 min and finally ramped to 90% in another 5 min gradient. Both solvent A(water) and B(ACN) contain 0.3% formic acid. The resolved peptides were detected by an ESI-TOF spectrometer (LCT premier, Micromass/Waters, Milford, Mass.). The capillary voltage for electrospray was set at 3000V, sample cone at 75V. Desolvation was accelerated by maintaining the desolvation temperature at 150° C. and source temperature at 100° C. The desolvation gas flow was 300 L/h. The data was acquired in “V” mode and the TOF was externally calibrated by Sodium Iodide and Cesium Iodide mixtures. The instrument was controlled by MassLynx 4.0 software.

### Protein Identification

Digested peptide mixtures from NPS RP HPLC collection or in-gel digestion were separated in the same manner as described above. The resolved peptides were analyzed on an LTQ mass spectrometer with an ESI ion source (Thermo, San Jose, Calif.). The capillary temperature was 175° C., spray voltage was 4.2 kV and capillary voltage is 30V. The normalized collision energy was set at 35% for MS/MS. MS/MS spectra were searched using SEQUEST algorithm incorporated in Bioworks software (Thermo) and the Swiss-Prot human protein database. One mis-cleavage is allowed during the database search. Positive protein identification was accepted for a peptide with Xcorr of greater than or equal to 3.0 for triply-, 2.5 for doubly- and 1.9 for singly charged ions.

### Results and Discussion:

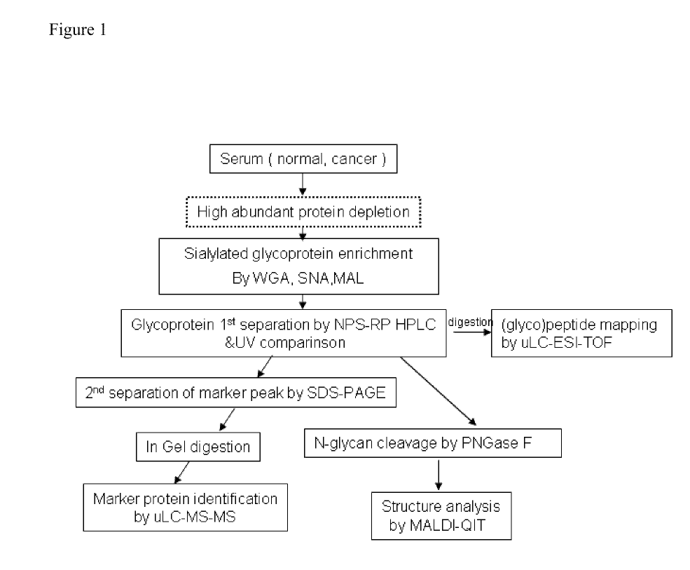

The analytical strategy used in this work is outlined in FIG. 1. Glycoproteins containing sialic acid were enriched using WGA, SNA and MAL affinity columns separately. Part of the serum sample was depleted before the lectin extraction step for the detection of medium and low abundant proteins. The lectin enriched fractions were fractionated by NPS-RP-HPLC and the eluting proteins were detected with UV absorption detection. The altered peaks between normal and cancer samples were further separated by SDS-PAGE followed by in gel digestion. The potential marker proteins were identified by peptide sequencing using μLC-MS/MS. N-glycans were cleaved from target glycoproteins by PNGase F. The structures of oligosaccharides released were analyzed by a hybrid ion trap T of mass spectrometer. Glyco-peptide mapping was performed using a LC-ESI-TOF MS in order to study the change in the structure of the isoforms and the extent of glycosylation in target glycoproteins in cancer serum. Three normal serum and three cancer serum samples were analyzed in this work and reproducible results were obtained.

### Analysis of Depleted Serum Sample

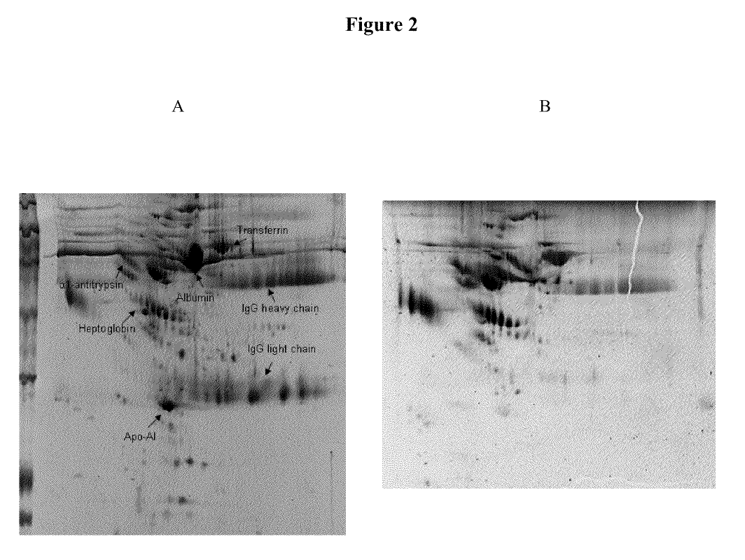

The serum proteome is dominated by a few highly abundant proteins that constitute about 90% of the total protein content of serum. These proteins severely interfere with the quantification and identification of proteins of low abundance (Echan et al., Proteomics 2005, 5, 3292-3303). In FIG. 2(a), 5 μl serum sample (250 μg) was loaded onto a 2-D gel which has reached the loading capacity of the gel. Only high abundant proteins can be detected and their presence masks the detection of low abundant proteins. Most of the high abundant proteins are indicated as glycosylated as shown in FIG. 2(b) where the gel is stained with glycoprotein dye. Although albumin is not a glycoprotein, it binds to other glycoproteins so that partial binding to lectins occurs and it is stained by the glycoprotein dye. Since many important marker proteins are detected in low concentration in biological samples, removing the high abundant proteins may be a critical strategy for serum biomarker discovery.

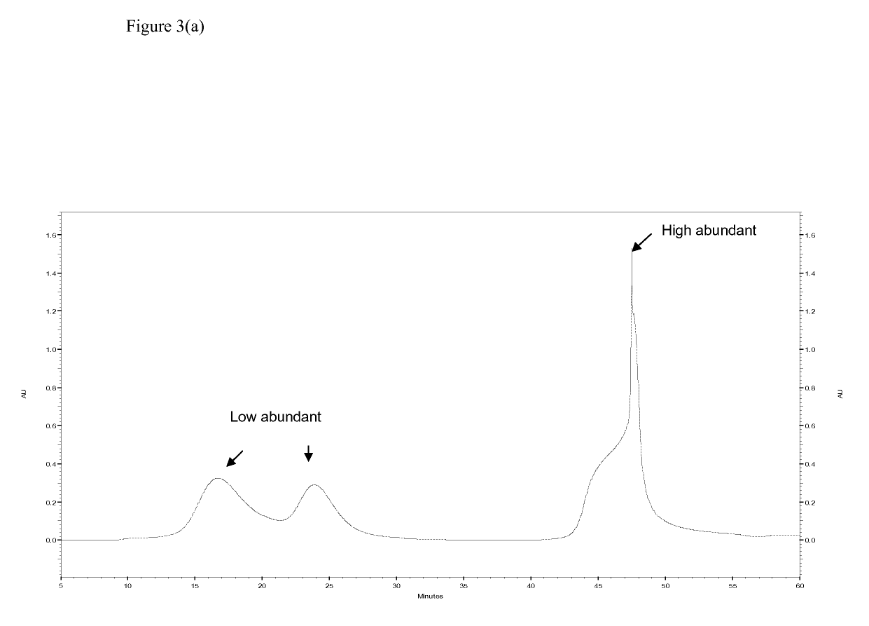

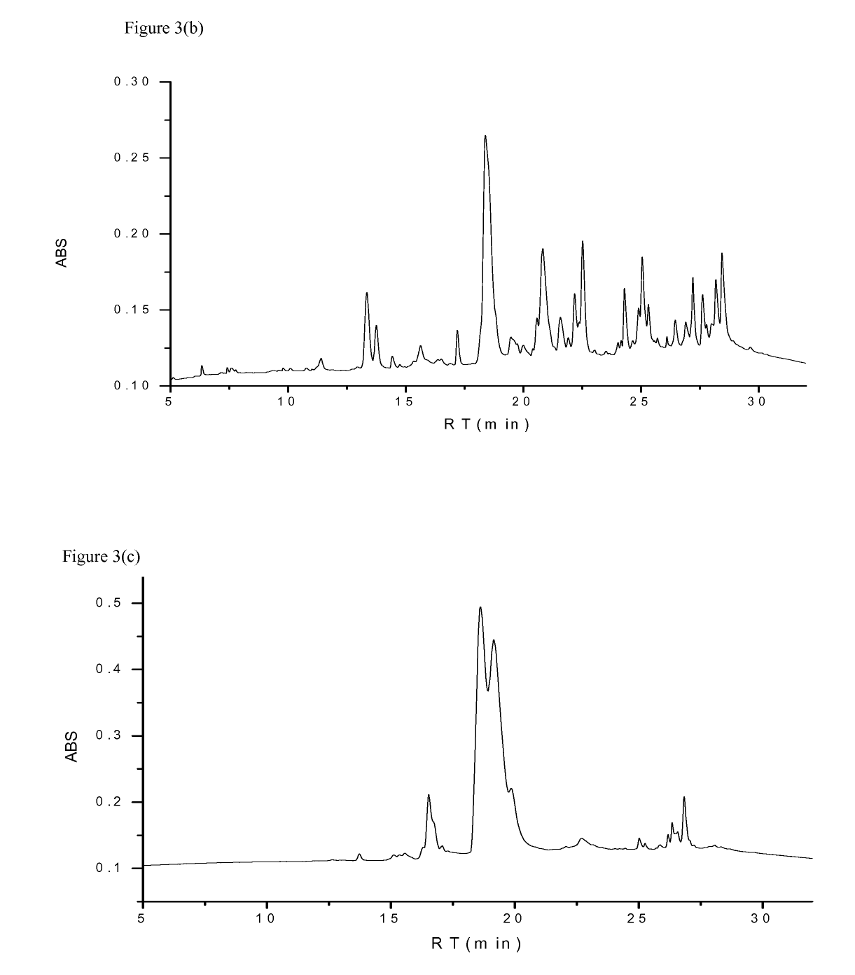

In this study, twelve highly abundant proteins (albumin, IgG, α1-antitrypsin, IgA, IgM, transferring, haptoglobin, α1-acid glycoprotein, α2-macroglobin, HDL (apolipoproteins A-I&A-II) and fibrinogen) were removed using an affinity column based on avian antibody (IgY)-antigen interactions. FIG. 3(a) shows the chromatogram of the binding and washing process of 125 μl of human serum. The protein assay result indicates that around 7% of total protein was retained in the low abundant fraction. From the LC separation of 20 μg protein from the low abundant (FIG. 3b) fraction and 15 μg protein from the high abundant fraction (FIG. 3c) using a C18 NPS-RP column, it was observed that most of the high abundant proteins have been effectively removed except some fraction of the albumin. With removal of the highly abundant proteins, the remaining proteins can be identified over a relatively high dynamic range.

In order to compare the sialic acid glycoprotein expression between normal and pancreatic cancer serum, three lectins (WGA, MAL, SNA) were used to enrich sialic acid attached glycoproteins. These three lectins each bind different structural subclasses of these moieties. MAL could select glycoproteins containing NeuAc-Gal-GlcNAc with sialic acid at the 3 position of galactose (Wang et al., J Biol Chem 1988, 263, 4576-4585). SNA binds preferentially to sialic acid attached to terminal galactose in (a-2,6) and to a lesser degree, (a-2,3) linkage (Shibuya et al., Arch Biochem Biophys 1987, 254, 1-8). WGA can interact with some glycoproteins via sialic acid residues and it also binds oligosaccharides containing terminal N-acetylglucosamine (Bakry et al., J Pharmacol Exp Ther 1991, 258, 830-836). Proteins bound with WGA were eluted by 0.5 M N-acetyl-glucosamine and proteins bound with SNA and MAL were eluted by 0.3 M Lactose. The protein assay indicated that 5-10% of the protein content is extracted by the lectin affinity columns. The parallel application of these three lectins gave a complete profile of sialylated glyco-conjugates with heterogeneous structures and it also provides information on the distribution of the sialylic glycoproteins with different sub-structures.

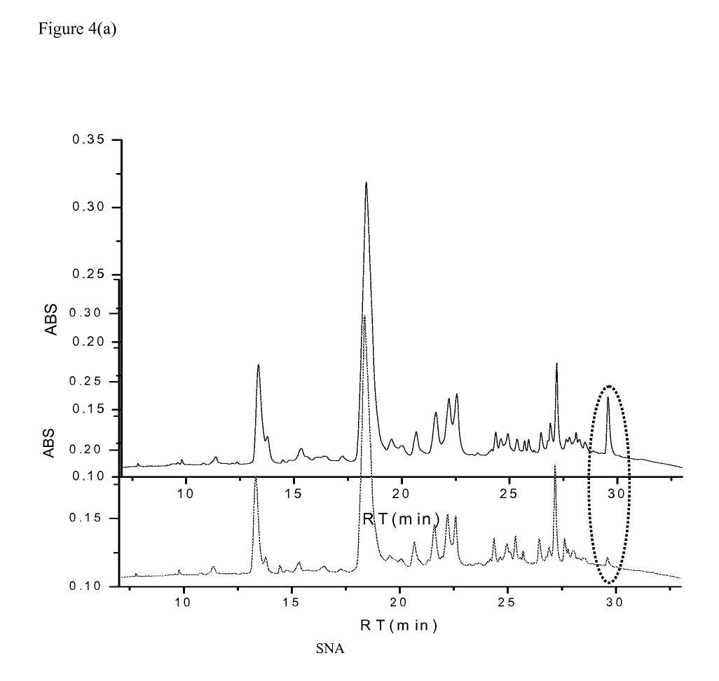

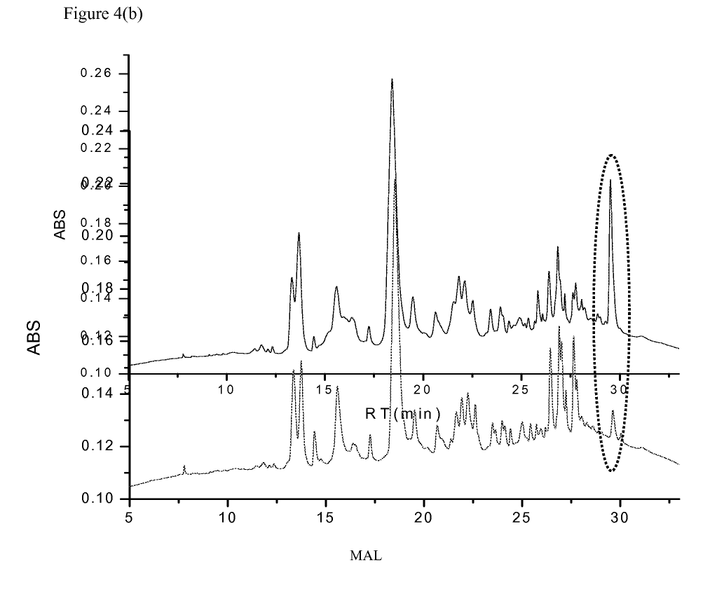

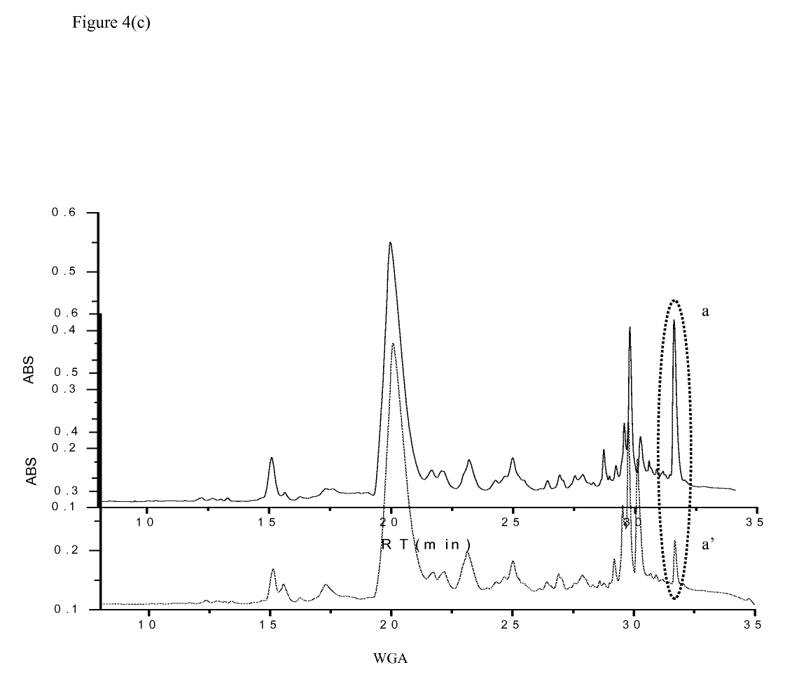

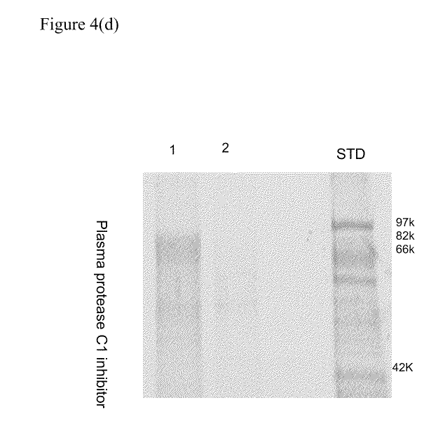

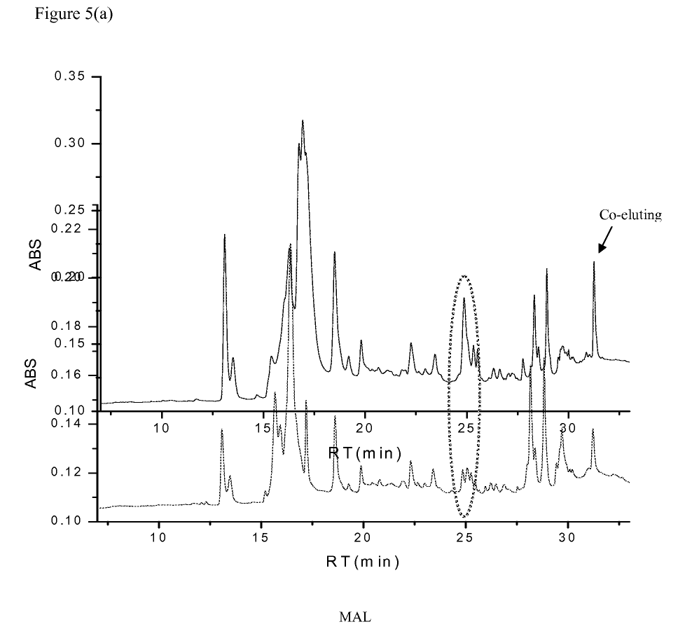

The enriched glycoproteins were further separated using a nonporous reversed phase (NPS-RP) C18 column where rapid separation (<35 min) and relatively high resolution of intact proteins can be achieved. The high complexities of the serum glycol sample make it difficult to achieve complete separation by a single dimension fractionation. Further separation of the fraction of interest was performed by SDS-PAGE gel electrophoresis. FIG. 4(a-c) shows the separation of each lectin enriched glycoprotein fraction from normal and pancreatic cancer serum samples by NPS-RP HPLC. About 130 glycoproteins were identified by LC-MS/MS from the WGA enriched fraction after fractionation by RP HPLC, which is comparable with the number of proteins identified by single LC-MS/MS in previous work (Yang et al., Proteomics 2005, 5, 3353-3366). The advantage of using the protein pre-fractionation strategy instead of the shotgun proteomics approach is that the information available from the intact protein including molecular weight and pI is available for protein identification including isoform characterization as shown in FIG. 4d where several isoforms may be present.

The use of UV absorption of intact proteins with NPS-RP separations provides a means to quantify the expression of glycoproteins of a given structure. A comparison of the peaks in the UV chromatogram of lectin extracted glycoproteins between normal and cancer samples show a very similar pattern between the two samples. Provided that the same amount of sample is loaded onto the column in each case, the method is highly reproducible. One peak (peak a) in each lectin extracted chromatogram (FIG. 4 a-c) eluted at around 30 min and shows an obvious quantitative difference in expression between normal and cancer sample. The SDS-PAGE gel separation (see FIG. 4 (d)) of this target peak indicates two bands at around 60 k and 85 k. After in gel tryptic digestion, both of these two bands were identified as plasma protease C1 inhibitor. The theoretical Mr of this protein is 55 k and it is one of the most heavily glycosylated plasma proteins. There are seven theoretical N-glycosylation sites on the sequence of this protein. The “poor focus” of the bands in the 1-D gel of FIG. 4d is due to the heavy modification of this protein. The intensity difference of the gel bands also confirms that this protein is down-regulated in cancer serum.

Table 1 lists the normalized peak areas of the peak for plasma protease C1 inhibitor observed in FIG. 4 for each lectin extraction via NPS RP-HPLC with UV absorption detection. As indicated, plasma protease C1 inhibitor in cancer serum is down-regulated 5.6×, 5.0×, 4.7× in SNA, MAL and WGA enriched fractions respectively relative to normal serum. By comparing the peak intensity among these three lectins, it was found that quantitatively more sialic acid tends to attach to the terminal galactose in (a-2,3) position than in a-2,6 position since the expression of this protein in the MAL extracted fraction is higher than in the SNA extracted fraction as shown in Table 1. The expression in the WGA extracted fraction is highest since WGA is not only specific to terminal sialic acid but also interacts with terminal N-acetylglucosamine. The relatively small change in the ratio of these three lectin enriched sialylated C1INH indicates that the change might be due to either altered levels of protein expression or altered sialylation on this protein while the distribution of sub-class structure does not obviously change.

Protease C1 inhibitor may play a potentially crucial role in regulating important physiological pathways including complement activation, blood coagulation, fibrinolysis and the generation of kinins. It has been reported that N-glycans of this protein from patients with a heterozygous genetics deficiency were small, highly charged and lacked sialidase releasable N-acetylneuraminic acid (Yu et al., supra) and C1INH plays a direct role in leukocyte-endothelial cell adhesion where the activity is mediated by carbohydrate (Zhang et al., Biochim Biophys Acta 2004, 1739, 43-49; Cai et al., J Immunol 2005, 174, 6462-6466).

### Analysis of the Undepleted Serum Sample

The serum sample was also analyzed without depletion of the most highly abundant proteins. This fraction is also useful since many of these proteins may also play an important role in biological systems critical to the cancer progression. Also, the high concentration of these proteins in serum provides for more sensitive detection and improved quantitative analysis.

The sialic attached glycoproteins from non-depleted serum samples were enriched in the same fashion as in the depleted samples. FIG. 5 (a-c) shows the NPS-RP separation of enriched glycoproteins from non-depleted normal and cancer serum samples. Many low abundant peaks in FIG. 4(a-c) are suppressed by the presence of high intensity peaks as shown in FIG. 5 (a-c) which were identified as albumin, transferrine, α1-antitrypsin, etc. The marker peak identified in the depleted sample at a retention time around 30 min did not show an obvious change with the presence of the high abundant proteins. μLC-MS/MS analysis on the digests of this peak indicates that plasma protease C1 inhibitor co-elutes with alpha2-macroglobulin. Therefore the expression change of this protein is masked by the high levels of macroglobulin. In a comparison of the UV chromatogram between normal and cancer samples, one broad peak is consistently down-regulated in the cancer samples. A further separation using SDS-PAGE and identification by μLC-MS/MS indicates the presence of IgG isoforms. In FIG. 5 (d) the heavy chain shows up around 70 k and the light chain at 30 k Da in the gel. It was also observed that there are other low concentration proteins that co-elute with IgG at this retention time, although the IgG isoforms are the components that are responsible for the peak intensity decreases in the cancer sample. The normalized peak area data in Table 1 indicates that IgG from cancer serum is expressed 1.8×, 1.4× and 2.9× lower in SNA, MAL and WGA enriched sialic glycoforms respectively. The change in WGA extracted glycoforms is greater than the other two lectin extractions. This may be caused by the “co-down-regulation” of the glyco isoforms with terminal N-acetylglucosamine and sialic acid since WGA interacts with both of these two structures. Therefore, the glycosylation pattern on IgG is different between normal and cancer serum. The reduced level of sialylated IgG in cancer serum indicates that glycosylation on this protein may be related to the immune response of the cancer cell. In addition, the higher intensity observed for the SNA selected glycoform than in MAL shows that there are more sialic acids attached to the terminal galactose in α-2,6 position than in α-2,3 position.

### Isoform Change of α1-Antitrypsin in Cancer Serum

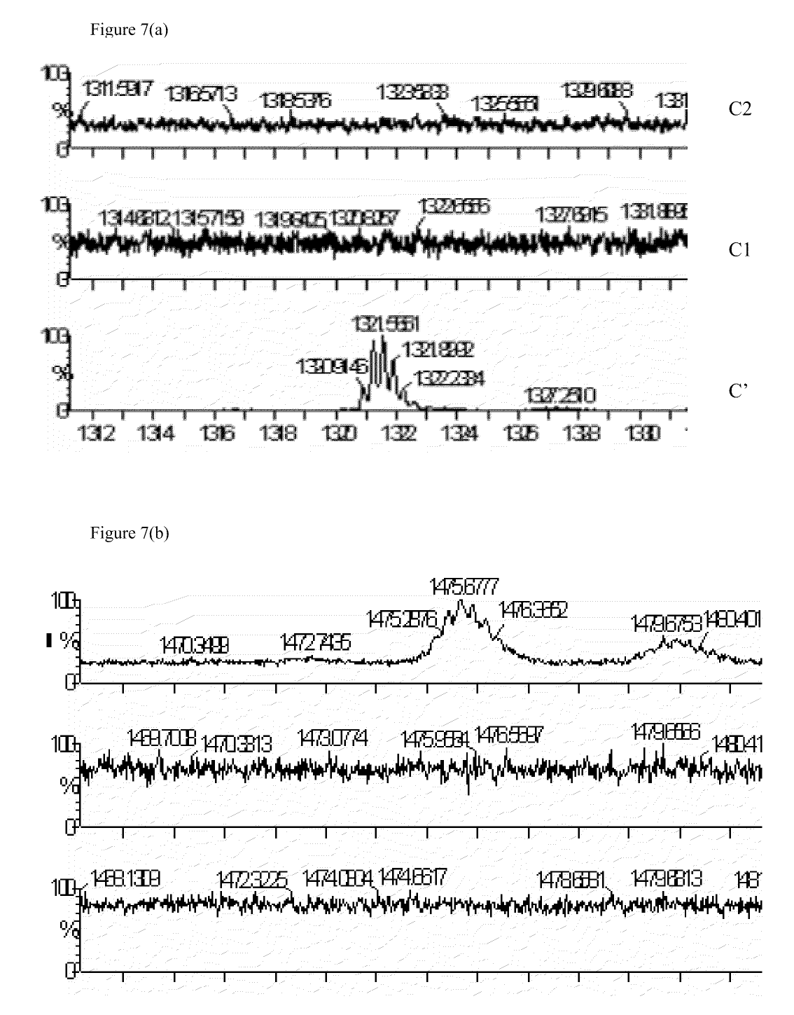

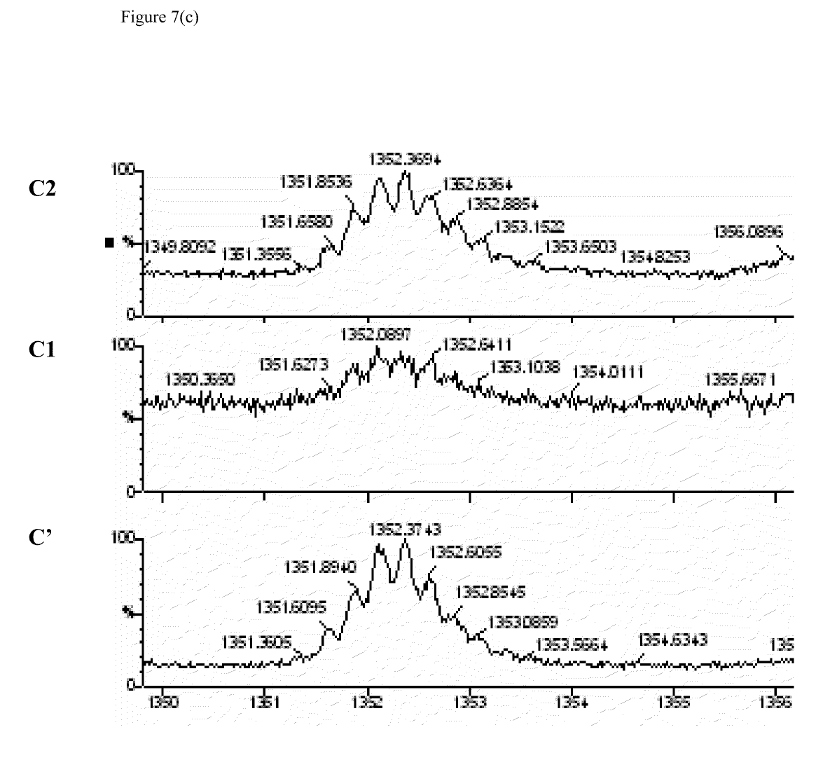

A comparison of the structure of glycosylated sites in serum proteins has provided some information about the factors affecting glycosylation site occupancy. A previous study of plasma α1-antitrypsin from congenital disorders of glycosylation type 1 patients has shown that the sites of glycosylation that are not occupied is not random under conditions of decreased glycosylation capacity and the asparagine residues are preferentially glycosylated in the order 46>247>83 in mature under-glycosylated forms (Mills et al., Glycobiology 2003, 13, 73-85). In this study, the peak in the UV chromatogram (see FIG. 5) of α1-antitrypsin enriched by WGA was observed to change in shape in the pancreatic cancer serum. The two peaks of this protein appearing in normal serum were labeled as c1 and c2 and the one peak in cancer serum was labeled as c′. The result of glycopeptide mapping of c1, c2 and c′ indicates that there is change in the glycosylation site occupancy of α1-antitrypsin in the pancreatic cancer serum. The tryptic digests of these three peaks were analyzed by μLC-ESI-TOF with the LCT mass spectrometer (FIG. 7). The multiple charge capability of ESI enables one to detect the high mass ions (>3000 m/z) in the low mass range (<1500 m/z), which provides improved sensitivity and mass accuracy.

The changes in glycosylation occupancy are shown in FIGS. 7 a-c as a pattern of multiple charged peaks. Table 3 lists all the glycopeptides detected by μLC-ESI-TOF and the corresponding glycan structures were determined by QIT-TOF. The dominant glycan structure on this protein is determined as a biantennary structure with two sialic acids attached. The results show that for asparagine 247 this glycan was only observed in the cancer serum (FIG. 7a, #3 in Table 3) and on N83, this glycan only appears in peak c2 of the normal serum (FIG. 7b, #6 in Table 3), while as shown in FIG. 7c (#8 in Table 3), this glycan was detected on N46 of both sample. As shown in Table 3, N46 was mainly attached with a biantennary and fucosylated biantennary glycan, N83 was mainly modified with bi- and tri-antennary glycans while N247 was modified with a biantennary glycan. Comparing the site occupancy among peaks c1, c2 and c′, it was found that N247 and part of N46 was occupied in peak c1, in peak c2, N83 and N46 was fully occupied, while in peak c′, N247 and N46 was occupied. This result shows that in WGA enriched α1-antitrypsin, N83 was deglycosylated in pancreatic cancer serum, while in normal serum this protein is not fully glycosylated at three sites simultaneously. N46 is most easily occupied. The change of glycosylation isoforms of α1-antitrypsin indicates a decrease in glycosylation capacity in cancer serum and the efficiency of glycosylation site occupancy is related to structural features at each site. The results suggest that N83 is most easily deglycosylated while N46 is preferentially occupied. In addition, it demonstrates the capability of using RP-HPLC coupled with μLC-ESI-TOF to detect the glycosylation isoform changes between samples.

### Glycan Structure Analysis of Target Proteins

In this study, the endoglycosidase PNGase F was used to remove almost all types of N linked (Asn linked) glycosylation from the protein of interest. MALDI has proved to be the most effective method to ionize N-link carbohydrates since it does not require the carbohydrates to be derivatized (Harvey, Mass Spectrom Rev 1999, 18, 349-450). Ion trap instruments have the capability to perform multiple successive stages of fragmentation which allows probing the details of carbohydrate structure. The interface of MALDI to the MALDI-quadrupole iontrap-ToF provides a means of performing multiple stages of CID with high mass accuracy and resolution (Fountain et al., Rapid Commun Mass Spectrom 1994, 8, 407-416; Ding et al., Proc. Int. Soc. Optical Eng. 1999, 3777, 144; Chien et al., Rapid Commun Mass Spectrom 1993, 7, 837-843; Doroshenko et al., Mass Spectrom 1998, 33, 305-318). This instrument was used to study the released glycan from the target protein where the carbohydrate could be studied with fragmentation up to MS4 (Demelbauer et al., Rapid Commun Mass Spectrom 2004, 18, 1575-1582; Ojima et al., Mass Spectrom 2005, 40, 380-388). MALDI in this instrument produced a strong [M+Na]+ ion from neutral N-linked glycans as shown in Table 2. Because of the extended transit time from the MALDI target into the ion trap, some in-source fragmentation has been observed (Harvey et al., Rapid Commun Mass Spectrom 2004, 18, 2997-3007). However, sialylated glycans showed very extensive fragmentation with almost complete loss of sialic acid, which is common with spectra recorded with reflectron-TOF instruments.

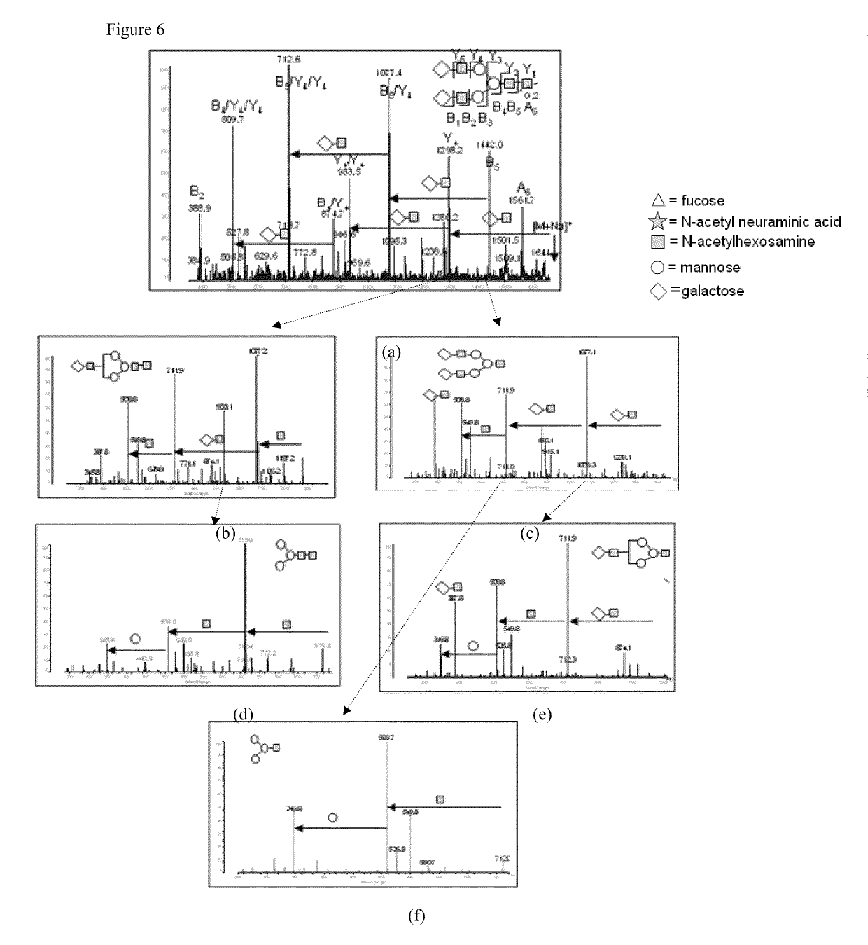

FIG. 6 shows the assignment of the structure of a biantennary glycan cleaved from α1-antitrypsin using the QIT. As shown in FIG. 6(a), the MS2 spectra of the biantennary glycan was dominated by ions produced of Y-type cleavages of terminal GlcNAc residues from the reducing end and loss of GlcNAc-Galactose residue from the non-reducing end. One strong cross-ring cleavage was observed at 1562 m/z in the MS2 spectrum. It was formed by cleavage at the (0,2) position of the sugar ring. The subsequent multiple stages (MS3 and MS4) of fragmentation clearly explain the ion formation pathway. As shown in FIG. 6 (b-f), the main fragment pathway is [M+Na]+(1663 m/z))-B5 (1442 m/z) or Y4 (1298 m/z)-B5/Y4(1077 m/z)-B5/Y4/Y4 (712 m/z)-B4/Y4/Y4 (509 m/z). Table 2 lists the detected mass and assigned structures of the glycans attached to IgG, α1-antitrypsin and plasma protease C1 inhibitor as determined using the MALDI-QIT-TOF MS. Terminal sialic acid was not detected because of the in-source fragmentation, so the acidic glycans were detected as the neutral type. A variety of approaches have been developed to modify the carboxyl group such as methyl esterification (Powell et al., Rapid Commun Mass Spectrom 1996, 10, 1027-1032), permethylation (Juhasz et al., Am. Soc. Mass Spectrom. 1992, 3, 785-796) and amidation (Sekiya et al., Anal Chem 2005, 77, 4962-4968). The main types detected from these three proteins are biantennary glycan, fucosylated biantennary glycan and triantennary glycan. Considering the lectins used in this work mainly interact with sialic acid attached to terminal galactose in (a-2,6) and (a-2,3) position as well as terminal N-acetylglucosamine, it is expected that these glycoforms are preferably selected by WGA, MAL and SNA. For the protease C1 inhibitor, there might be more glycan diversity attached to this protein since it is very heterogeneous on the gel image. Because of its low abundance in the serum sample, only the dominant structure type (biantennary) is assigned. The detection of oligosaccharides released from target proteins using this strategy allows the potential ability to compare different carbohydrates between samples.

TABLE 1
Normalized UV peak areas of differentially expressed
proteins enriched by three different lectins.

| Lectin specific structure | 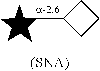 | 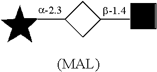 | 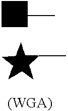 |
|---|---|---|---|
| Plasma protease | Acc#. P05155 | | |
| C1 inhibitor | | | |
| Normal | 0.0075 | 0.0117 | 0.0679 |
| Cancer | 0.0013 | 0.0023 | 0.0146 |
| change | 5.6 | 5.0 | 4.7 |
| IgG isoforms | | | |
| Normal | 0.0686 | 0.0078 | 0.2560 |
| Cancer | 0.0375 | 0.0054 | 0.0886 |
| change | 1.8 | 1.4 | 2.9 |

TABLE 2
Neutral glycan structures assigned from three target
proteins using MALDI-QIT.

| | |
|---|---|
| Detected | |
| Mass | Proposed |
| (Na⁺) | structure |

IgG

| | |
|---|---|
| 1810.1 | 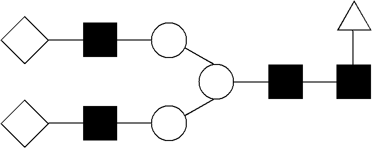 |
| 1648.0 | 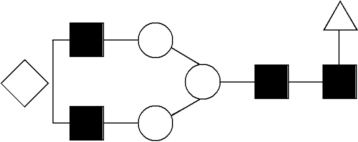 |
| 1486.0 | 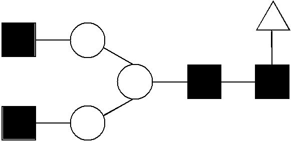 |
| 1445.1 | 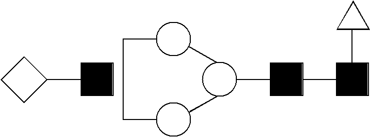 |
| 1663.3 | 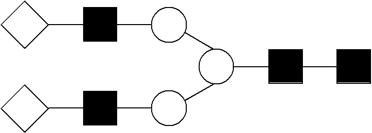 |

a₁-antitrypsin

| | |
|---|---|
| 1663.0 | 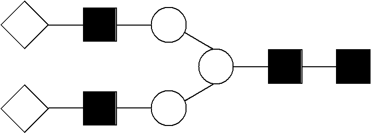 |
| 1810.2 | 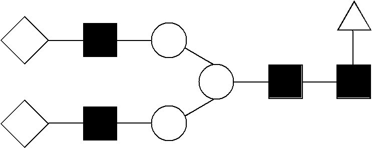 |
| 2028.0 | 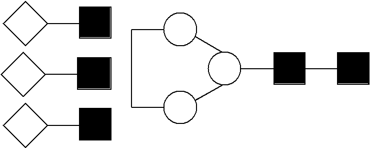 |

Plasma protease C1 inhibitor

| | |
|---|---|
| 1663.4 | 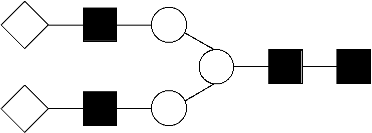 |

TABLE 3
Glycopeptide mapping for tryptic digests of α1-
antitrypsin using μLC-ESI-TOF.

| | | | Glyco- | | | |
|---|---|---|---|---|---|---|
| | Detected | | peptide | Peptide | Glycan | Detect |
| | mass | charge | mass | id | structure | location |
| 1 | 1437.3 | 4+ | 5745.17 | 244-274¹ N247 | 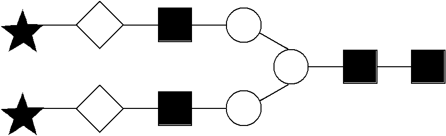 | Peak C Peak C1 |
| 2 | 1150.0 | 5+ | 5744.96 | 244-274 N247 | 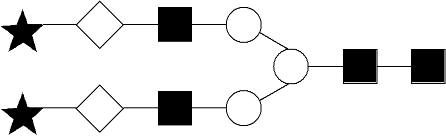 | Peak C′ Peak C1 |
| 3 | 1320.9 | 3+ | 3959.68 | 244-259 N247 | 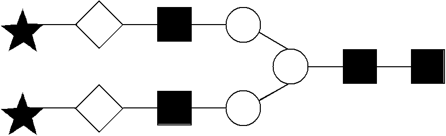 | Peak C′ |
| 4 | 1223.6 | 3+ | 3668.87 | 244-259 N247 | 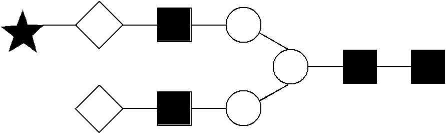 | Peak C′ Peak C1 |
| 5 | 1639.1 | 4+ | 6552.37 | 70-101 N83 | 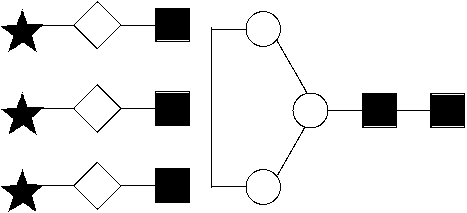 | Peak C2 |
| 6 | 1475.1 | 4+ | 5896.37 | 70-101 N83 | 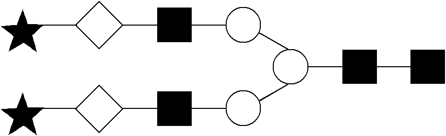 | Peak C2 |
| 7 | 1278.7 | 4+ | 5110.77 | 40-69 N46 Met-ox² | 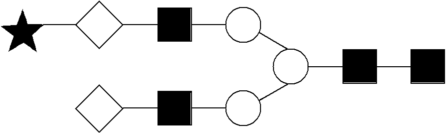 | Peak C′ Peak C2 |
| 8 | 1351.6 | 4+ | 5402.37 | 40-69 Met-ox N46 | 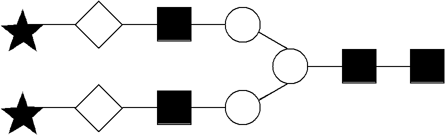 | Peak C′ Peak C1 (trace) Peak C2 |
| 9 | 1387.8 | 4+ | 5547.17 | 40-69 N46 Met-ox | 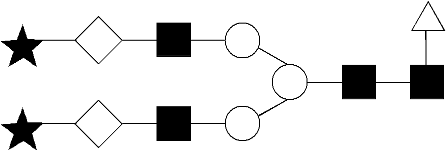 | Peak C′ Peak C2 |

1. peptide 244-274 result from one miscleavage.
2. Met-ox = methionine oxidation.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A system, comprising
a) a lectin affinity chromatography apparatus; and
b) a liquid chromatography apparatus configured to receive a protein sample separated by said lectin affinity chromatography apparatus.

2. The system of claim 1, wherein said lectin affinity chromatography apparatus comprises a lectin affinity column selected from the group consisting of wheat Germ Agglutinin, Elderberry lectin, and Maackia amurensis lectin.

3. The system of claim 1, wherein said liquid chromatography apparatus comprises a non-porous reverse phase HPLC apparatus.

4. The system of claim 1, wherein said system further comprises an apparatus for removal of highly abundant serum proteins.

5. The system of claim 4, wherein said apparatus is an IgY-12 proteome partitioning column.

6. The system of claim 5, wherein said IgY-12 proteome partitioning column is configured for the removal of albumin, IgG, α1-antitrpsin, IgA, IgM, transferring, haptoglobin, α1-acid glycoprotein, α2-macroglobin, apolipoproteins A-I and A-II and fibrinogen in a single step.

7. The system of claim 1, further comprising an apparatus for performing polyacrylamide gel electrophoresis.

8. The system of claim 1, further comprising a mass spectrometry apparatus.

9. The system of claim 8, wherein said mass spectrometry apparatus is selected from the group consisting of a MALDI-TOF mass spectrometer, a QIT MALDI quadrupole ion trap-ToF spectrometer, an ESI-TOF mass spectrometer, and an ESI-LTQ mass spectrometer.

10. A method, comprising:
a) treating a protein sample with a lectin affinity chromatography apparatus under conditions such that said lectin affinity chromatography apparatus enriches said protein sample for glycosylated proteins to generate a glycosylated protein enriched sample; and
b) separating said glycosylated protein enriched sample with a liquid chromatography apparatus to generate a separated glycosylated enriched protein sample.

11. The method of claim 10, wherein said lectin affinity chromatography apparatus comprises a lectin affinity column selected from the group consisting of wheat Germ Agglutinin, Elderberry lectin, and Maackia amurensis lectin.

12. The method of claim 10, wherein said liquid chromatography apparatus comprises a non-porous reverse phase HPLC apparatus.

13. The method of claim 10, further comprising the step of prior to said treating with said lectin affinity chromatography apparatus, the step of treating said protein sample with an apparatus for removal of highly abundant serum proteins.

14. The method of claim 13, wherein said apparatus is an IgY-12 proteome partitioning column.

15. The method of claim 14, wherein said IgY-12 proteome partitioning column removes albumin, IgG, α1-antitrpsin, IgA, IgM, transferring, haptoglobin, α1-acid glycoprotein, α2-macroglobin, apolipoproteins A-I and A-II and fibrinogen in a single step.

16. The method of claim 10, further comprising the step of performing polyacrylamide gel electrophoresis on said separated glycosylated enriched protein sample.

17. The method of claim 10, further comprising the step of performing mass spectrometry on said separated glycosylated enriched protein sample.

18. The method of claim 17, wherein said mass spectrometry is selected from the group consisting of MALDI-TOF mass spectrometry, QIT MALDI quadrupole ion trap-ToF mass spectrometry, ESI-TOF mass spectrometry, and ESI-LTQ mass spectrometry.

19. The method of claim 10, wherein said sample is from a subject diagnosed with cancer.

20. A method of comparing protein profile maps, comprising
a) treating first and second protein samples with a lectin affinity chromatography apparatus under conditions such that said lectin affinity chromatography apparatus enriches said protein sample for glycosylated proteins to generate first and second glycosylated protein enriched sample;
b) separating said first and second glycosylated protein enriched samples with a liquid chromatography apparatus to generate first and second separated glycosylated enriched protein samples;
c) analyzing said first and second separated glycosylated enriched protein samples with a mass spectrometry apparatus to generate first and second protein profile maps; and
d) comparing said first and second protein profile maps.

21. The method of claim 20, wherein said first protein sample is from a subject diagnosed with cancer and wherein said second protein sample is from a cancer free subject.

22. The method of claim 20, further comprising the step of identifying proteins that are differentially expressed in said first protein sample relative to said second protein sample.

23. The method of claim 20, further comprising the step of identifying proteins with altered glycosylation patterns in said first protein sample relative to said second protein sample.

24. A method of diagnosing cancer in a subject, comprising: identifying an altered level of expression of a cancer marker selected from the group consisting of plasma protease C1 inhibitor and IgG in a sample from said subject relative to the level in a cancer-free subject.

25. The method of claim 24, wherein said cancer marker is expressed at a lower level in a subject with cancer relative to the level in a cancer-free subject.

26. The method of claim 24, wherein said sample is serum.

27. The method of claim 24, wherein said cancer is pancreatic cancer.

28. The method of claim 24, wherein said identifying an altered level of expression of said cancer marker comprises identifying an altered level of expression of cancer marker RNA.

29. The method of claim 24, wherein said identifying an altered level of expression of said cancer marker comprises identifying an altered level of expression of cancer marker polypeptide.

30. A method of diagnosing cancer in a subject, comprising: identifying an altered glycosylation pattern of α1-antitrypsin a sample from said subject relative to the glycosylation pattern of said α1-antitrypsin in a cancer-free subject.

31. The method of claim 30, wherein said identifying an altered glycosylation pattern of α1-antitrypsin comprises analyzing said glycosylation pattern with mass spectrometry.

32. The method of claim 30, wherein said identifying an altered glycosylation pattern of α1-antitrypsin comprises analyzing said glycosylation pattern with a labeled lectin.

33. The method of claim 30, wherein said identifying an altered glycosylation pattern of α1-antitrypsin comprises analyzing said glycosylation pattern with a glycosylation specific antibody.

34. The method of claim 30, wherein said identifying an altered glycosylation pattern of α1-antitrypsin comprises analyzing said glycosylation pattern with a glycosylation specific reagent.
